(19) United States
(12) Patent Application Publication
(10) Pub. No.: US 2016/0183872 A1
(43) Pub. Date: Jun. 30, 2016

(54) MULTI-MODE ACTIVE ORTHOTIC SENSOR

(71) Applicants: Robert W. HORST, San Jose, CA (US); John C. WESTMORELAND, San Jose, CA (US); Charles D. REMSBERG, Mountain View, CA (US)

(72) Inventors: Robert W. HORST, San Jose, CA (US); John C. WESTMORELAND, San Jose, CA (US); Charles D. REMSBERG, Mountain View, CA (US)

(21) Appl. No.: 14/932,796

(22) Filed: Nov. 4, 2015

Related U.S. Application Data
(63) Continuation of application No. 13/709,832, filed on Dec. 10, 2012, now abandoned.
(60) Provisional application No. 61/569,188, filed on Dec. 9, 2011.

(51) Int. Cl. A61B 5/00; A61B 5/103; A61F 5/01

(52) U.S. Cl. CPC A61B 5/4851; A61F 5/0102; A61B 5/4836; A61B 5/0031; A61B 5/1038; A61B 2090/064

(57) ABSTRACT

A general-purpose force sensor, which can be used with an orthotic device, is provided utilizing both resistive and capacitive techniques for improved accuracy and reliability compared to either type of sensor alone. The system can detect internal fault conditions and continues to operate correctly despite the failure of one of the sensors. The sensor can be self-calibrating to give accurate readings despite changes in the physical properties of the sensing elements over time.

### CROSS REFERENCE TO RELATED APPLICATIONS

This application is a continuation of U.S. patent application Ser. No. 13/709,832, filed on Dec. 10, 2012, titled “Orthotic Device Sensor,” Publication No. US-2013-0165817-A1, which claims the benefit of U.S. Provisional Application No. 61/569,188 filed on Dec. 9, 2012 and titled “Orthotic Device Sensor,” which is hereby incorporated by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference. The application, for example, incorporates in entirety by this reference U.S. Pat. No. 8,052,629, filed Feb. 6, 2009, of Jonathan Smith et al., entitled “Multi-Fit Orthotic and Mobility Assistance Apparatus,” U.S. Publication No. 2010/0038983 filed Jan. 30, 2009, of Kern Bhugra et al., entitled “Actuator System with a Motor Assembly and Latch for Extending and Flexing a Joint,” U.S. Pat. No. 6,966,882 filed Nov. 6, 2003, of Robert Horst entitled “Active Muscle Assistance Device and Method;” and U.S. patent application Ser. No. 12/703,067, of Robert Horst, et al., entitled “Foot Pad Device and Method of Obtaining Weight Data,” filed on Feb. 9, 2010.

### FIELD

Embodiments of the present invention relate generally to orthotics, and more specifically to sensors for active orthotics.

### BACKGROUND

Wearable active orthotic devices developed by the Applicant can be used to amplify the residual intention to extend or flex a joint of patients recovering from neuromuscular deficiencies arising from conditions including stroke, traumatic brain injury and multiple sclerosis. The effectiveness of these devices is dependent on an accurate assessment of the intention of the patient to extend or flex a joint. In a knee augmentation device, the intention to extend the joint may be sensed by a foot pressure sensor. Similarly, extension or flexion of the elbow may be sensed by detecting pressure on the palm along with the rotation of the wrist. Sensors for active orthoses control the application of joint force; correct operation of these sensors is required to provide optimal therapy and avoid the possibility of injury.

### SUMMARY OF THE DISCLOSURE

The present invention relates to orthotics, and more specifically to sensors for active orthotics.

In some embodiments, a sensor is provided that detects internal fault conditions and continues to operate correctly despite the failure of one of the sensors.

In some embodiments, a sensor is provided that is self-calibrating to give accurate readings despite changes in the physical properties of the sensing elements over time.

In some embodiments, a sensor is provided with a unique ID that can be used to retrieve patient-specific information to reduce the time to begin therapy with a patient and to improve the accuracy of data collection and device configuration.

In some embodiments, an interconnection to the sensor is provided that is self-aligning and pulls apart under moderate force to avoid injury to the patient and damage to the sensing device and interconnect wiring.

In some embodiments, a general-purpose force sensor is provided utilizing both resistive and capacitive techniques for improved accuracy and reliability compared to either type of sensor alone.

In some embodiments, a sensor for measuring force is provided. The sensor can include a first capacitive layer assembly having a capacitance that varies with the force applied to the sensor, a second capacitive layer assembly having a capacitance that varies with the force applied to the sensor, and a resistive layer disposed between the first capacitive layer assembly and the second capacitive layer assembly, the resistive layer having a resistance that varies with the force applied to the sensor.

In some embodiments, the first capacitive layer assembly includes a first conductive layer, a first ground layer and a first capacitive layer disposed between the first conductive layer and first ground layer, and wherein the second capacitive layer assembly includes a second conductive layer, a second ground layer and a second capacitive layer disposed between the second conductive layer and second ground layer.

In some embodiments, the resistive layer is adjacent to both the first conductive layer and second conductive layer.

In some embodiments, the conductive layers are made of a conductive fabric or ink.

In some embodiments, the capacitive layer assemblies and resistive layer are integrally formed in a fabric sock.

In some embodiments, the capacitive layer assemblies and resistive layer are integrally formed in a fabric glove.

In some embodiments, the sensor further includes an external surface having antimicrobial properties.

In some embodiments, the sensor further includes a sensor interface, wherein the sensor interface is in electrical communication with the conductive layers and the ground layers.

In some embodiments, the sensor interface includes a processing unit configured to measure the capacitance of the capacitive layer assemblies and the resistance of the resistive layer.

In some embodiments, the sensor interface is proximate the capacitive layer assemblies and the resistive layer.

In some embodiments, the sensor interface includes an activation counter.

In some embodiments, the sensor interface includes a magnetic connector with a north pole connector and a south pole connector.

In some embodiments, the north pole connector and the south pole connector are electrically connected to the conductive layers and the ground layers.

In some embodiments, a method of self-calibrating a sensor for measuring force is provided. The method can includes providing a sensor having a capacitive layer assembly with a capacitance that varies with the force applied to the sensor and a resistive layer with a resistance that varies with the force applied to the sensor, determining when no force is being applied to the sensor, adjusting a capacitance sensor offset when no force is being applied to the sensor so that the force measured by the capacitive layer assembly is set to zero, determining when a high level of force is being applied to the sensor, and adjusting a resistance sensor gain when a high level of force is being applied to the sensor so that the force measured by the resistive layer is set to be substantially equal to the force measured by the capacitive layer assembly.

In some embodiments, a method of operating a sensor for measuring force after detection of a fault is provided. The method can include providing a sensor with a first capacitive layer assembly having a capacitance that varies with the force applied to the sensor, a second capacitive layer assembly having a capacitance that varies with the force applied to the sensor, and a resistive layer disposed between the first capacitive layer and the second capacitive layer, the resistive layer having a resistance that varies with the force applied to the sensor; detecting one or more fault conditions by measuring at least one of a capacitance and resistance of the capacitive layer assemblies and the resistive layer; identifying the nature of the fault condition based on the measurement of at least one of a capacitance and resistance of the capacitive layer assemblies and the resistive layer; identifying one or more predetermined capacitance and resistance measurements that are accurate and not affected by the fault condition based on the identified nature of the fault condition; and determining the force measured by the sensor based on the one or more predetermined capacitance and resistance measurements that are accurate and not affected by the fault condition.

In some embodiments, a method of assisting movement of a subject is provided. The method can include providing a sensor with at least one resistive layer and at least one capacitive layer assembly; detecting a residual intention of the subject to move by measuring a force with the resistive layer and the capacitive layer assembly; and assisting the subject with the intended movement by applying an assistive force to the subject with an actuator.

In some embodiments, the sensor is a foot sensor and the actuator is a knee orthotic device.

In some embodiments, the sensor is a hand sensor and the actuator is an elbow orthotic device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an embodiment of the invention in the form of an active knee orthosis.

FIG. 2 illustrates examples of an orthotic system superimposed on subjects with varying degrees of leg alignment.

FIG. 3 illustrates another embodiment of a mechanical linkage between the actuator and the body attachment orthosis.

FIG. 4 is a block diagram showing the electronics used to drive and control the active muscle assistance device.

FIG. 5 is flowchart showing the modes of operation of a muscle assistance device.

FIG. 6 is a flowchart of the modes of operation of a knee joint muscle assistance device.

FIG. 7 is a block diagram of an embodiment of one or more sensors used for detecting body movement.

FIGS. 8A-8F illustrate various layers that form embodiments of the sensor.

FIG. 9 illustrates the assembly and orientation of the sensor layers in an embodiment of the sensor.

FIG. 10A is a block diagram of an embodiment of a sensor.

FIG. 10B is a block diagram of an embodiment of a controller for use with the sensor.

FIGS. 10C-10F illustrate additional embodiments of the sensor in use with a variety of different devices.

FIGS. 11A-11G illustrate an embodiment of the connection of a printed circuit board with the sensor layers.

FIGS. 12A and 12B are tables illustrating embodiments of fault detection and continued operation of the orthotic device.

FIG. 13 is a flow chart of an embodiment of fault tolerant operation of a sensor.

FIG. 14 is a flow chart of an embodiment sensor auto-calibration.

FIG. 15 is a flow chart of an embodiment of sensor initialization and determining sensor end of life.

### DETAILED DESCRIPTION

### General Overview of a Knee Orthosis

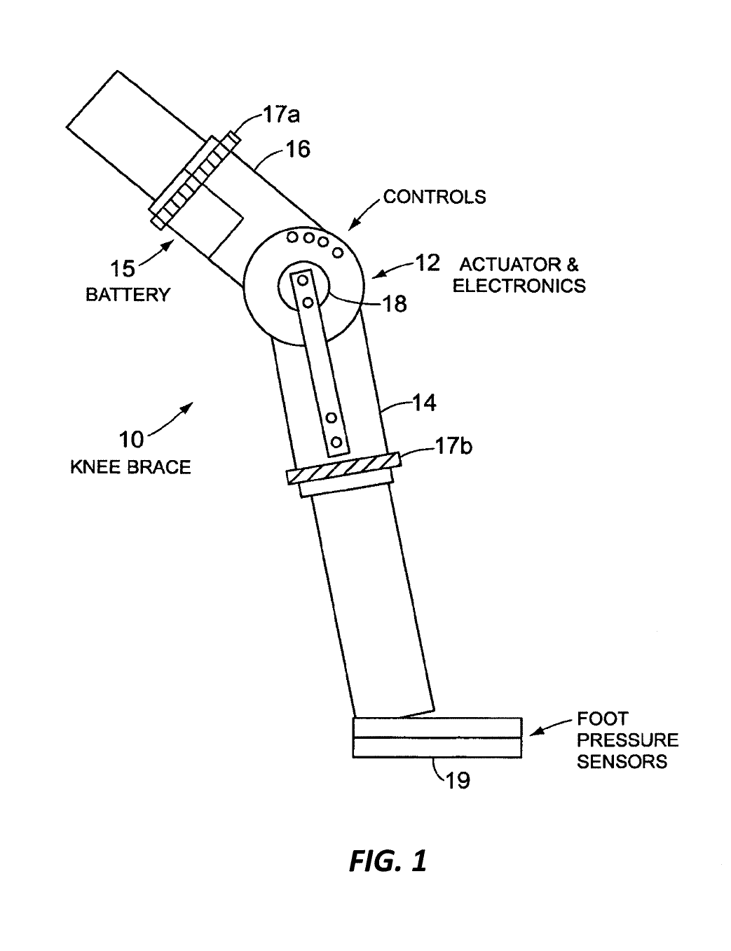

FIG. 1 shows an active muscle support orthosis according to one embodiment of the invention. The device is an active knee orthosis used to offload some of the stress from the quadriceps when extending or flexing the leg. For different parts of the body, other devices are constructed with a suitable shape, but the principles presented here apply by analogy to such devices. The device is particularly useful in helping someone with muscle weakness in the everyday tasks of standing, sitting, walking, climbing stairs and descending stairs. The support to the muscle is defined by the position of the actuator 12 applying force to the moving parts of the orthosis. Namely, as the actuator 12 rotates, and with it the moving (rigid) parts of the orthosis, the position of the actuator 12 defines the relative position of the joint and thereby supporting the corresponding muscle.

### Structure and Body Attachment

Each device provides assistance and/or resistance to the muscles that extend and flex one joint. In some embodiments, resistance can be provided to resist the force exerted by the muscles, and/or resistance can also be provided to resist or oppose the force of gravity. The device does not directly connect to the muscle, but is attached in such a way that it can exert external forces to the limbs. The device is built from an underlying structural frame, padding, and straps (not shown) that can be tightened to the desired pressure. The frame structure with hinged lower and upper portions (14 and 16) as shown is preferably made of lightweight aluminum or carbon fiber.

In this embodiment, the frame is attached to the upper and lower leg with straps held by hook and loop type fasteners (such as Velcro®) or clip-type connectors 17 or by a zipper type fastener. A soft padding material cushions the leg. The orthosis may come in several standard sizes, or a single size that may be adjusted to fit a variety of patients.

The attachment of the device to the body is most easily understood with respect to a specific joint, the knee in this case, which serves as an exemplary embodiment that can be adapted for use with other joints or body portions. The structural frame of the device includes a rigid portion above the knee connected to hinges 18 at the medial and lateral sides. The rigid structure goes around the knee, typically around the posterior side, to connect both hinges together. On the upper portion of the orthosis 16, the rigid portion extends up to the mid-thigh, and on the lower portion 14, it continues down to the mid-calf. In the thigh and calf regions, the frame extends around from medial to lateral sides around approximately half the circumference of the leg. The remaining portion of the circumference is spanned by straps that can be tightened with clips, laces or hook and loop closures. Understandably, this allows easier attachment and removal of the device. The rigid portion can be either on the anterior or posterior side. The number and width of straps can vary, but the straps must be sufficient to hold the device in place with the axis of rotation of the hinge in approximately the same axis as that of rotation of the knee. The hinge itself may be more complex than a single pivot point to match the rotation of the knee. In more general terms, in some embodiments the device has a frame that has a first structural portion that is attached to the body above or proximally the joint, a second structural portion that is attached to the body below or distally to the joint, and an articulating joint portion connecting the first structural portion with the second structural portion.

Cushioning material may be added to improve comfort. A manufacturer may choose to produce several standard sizes, each with enough adjustments to be comfortable for a range of patients, or the manufacturer may use a mold or tracing of the leg to produce individually customized devices.

As will be later explained in more detail, a microcontroller-based control system drives control information to the actuator, receives user input from a control panel function, and receives sensor information including joint position and external applied forces. For example, pressure information is obtained from the foot-pressure sensor 19. Based on the sensor input and desired operation mode, the control system applies forces to resist the muscle, assist the muscle, or to allow the muscle to move the joint freely.

The actuator 12 is coupled to the orthosis to provide the force needed to assist or resist the leg muscle(s). Although it is intended to be relatively small in size, the actuator may be located on the lateral side to avoid interference with the other leg. The actuator may also be located on an anterior region to allow a single orthotic device to be used no either the right or left leg of a patient. The actuator may be coupled to both the upper and lower portions of the structural frame to provide assistance and/or resistance with leg extension and/or flexion.

The actuator 12 may be structured to function as an electrostatic motor, linear or rotational (examples and implementations of electrostatic actuators can be found in U.S. Pat. Nos. 6,525,446, 5,708,319, 5,541,465, 5,448,124, 5,239,222). The actuator may also comprise one or more motors coupled to a lead screw or cable drive assembly or any other suitable motor.

The control panel may be part of the actuator or may be attached to another part of the structural frame with wires connected to the actuator. In some embodiments, buttons of the control panel can be of the type that can be operated through clothing to allow the device mode to be changed when the device is hidden under the clothes. In other embodiments, the device can be worn on top of clothing or can be worn directly on the skin and remain uncovered.

When the invention is applied to joints other than the knee, the same principles apply. For instance, a device to aid in wrist movement may have elastic bands coupling a small actuator to the hand and wrist. Joints with more than one degree of freedom may have a single device to assist/resist the primary movement direction, or may have multiple actuators for different degrees of freedom. Other potential candidates for assistance include the ankle, hip, elbow, shoulder and neck.

If the center of rotation of the actuator is located a distance away from the joint, a variety of coupling mechanisms can be used to couple the actuator to a portion of the orthosis on the other side of the joint. The coupling mechanism can be constructed using belts, gears, chains or linkages as is known in the art. These couplings can optionally change the ratio of actuator rotation to joint rotation.

In an embodiment using a linear actuator, the linear actuator has the stator attached to the femur portion of the orthosis and the slider is indirectly connected to the tibial part of the orthosis via a connecting cable stretched over a pulley. The center of rotation of the pulley is close to the center of rotation of the knee. With this arrangement, a second actuator may be used to oppose the motion of the first actuator if the device is to be used for resistance as well as assistance, or for flexion as well as extension.

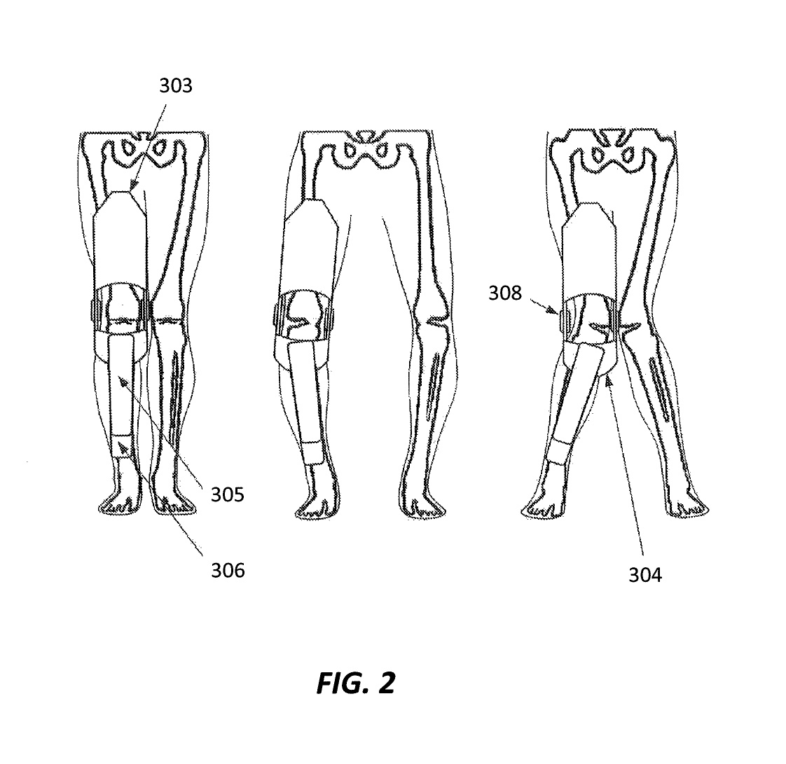

FIG. 2 illustrates embodiments of an orthotic system superimposed on subjects with varying degrees of leg alignment including nominal leg alignment as well as an extreme bowlegged subject and a knock-kneed subject.

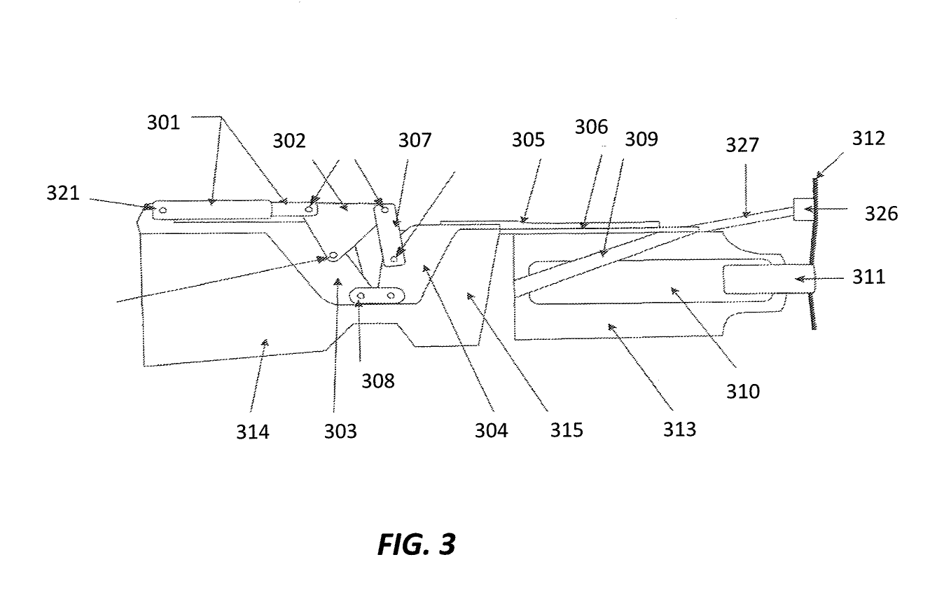

FIG. 3 illustrates a side-view diagram of an orthotic system according to an exemplary embodiment of the invention. In the illustrated embodiment, orthotic system 300 includes: linear actuator 301; bell crank 302; thigh orthotic structure 303; lower leg orthotic structure 304; tibia anterior structure 305; tibia posterior structure 306; connector link 307; hinge 308; tibia suspension system 309; lateral support structures 310; ankle suspension structure 311; footpad sensor system 312; lower leg textiles 313; thigh textile 314; upper shin textile 315; toe strap 326; and anti-foot drop system 327. However, this is given by way of example and not limitation, as the orthotic system described herein may include fewer or more components. Linear actuator 301 acts directly on a linkage point of a bell crank rocker arm 302. The linear actuator 301 is mounted on a pivot 321 at the upper most end of the thigh orthotic structure 303; however, other embodiments would include the linear actuator 301 being constrained on a fixed plane or fixed via pivot on any portion of the thigh orthotic structure 303 or lower leg orthotic structure 304 or other structural parts. Alternate embodiments would also include indirect actuation via an input link between the linear actuator 301 and the bell crank 302.

### Electronics and Control System Block Diagram and Operation

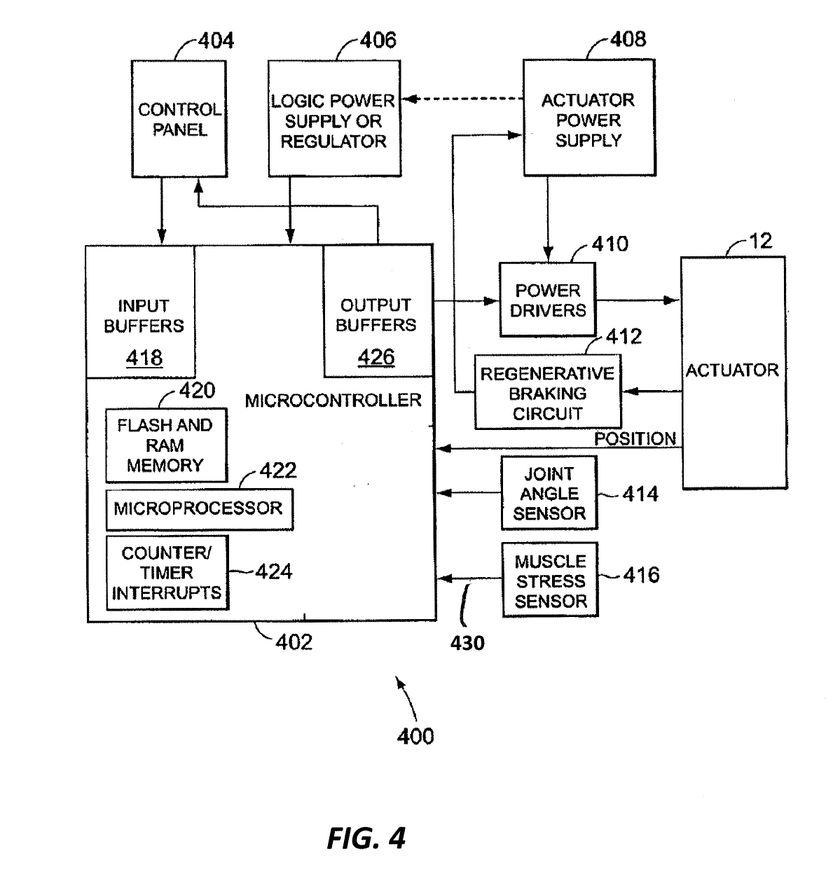

FIG. 4 is a block diagram showing the electronics and control system. The operation of the device may be controlled by a program running in a microcontroller 402. To minimize the physical size of the control system the microcontroller may be selected based on the scope of its internal functionality.

In this exemplary embodiment, the microcontroller 402 is coupled to a control panel 404 to provide user control and information on the desired mode of operation. The control panel includes a set of switches that can be read through the input buffers 418 of the microcontroller. The control panel also may have a display panel or lights to display information such as operational mode and battery state. The control panel also includes means to adjust the strength of assistance and resistance in order to customize the forces to the ability of the user. Another embodiment of the control panel is a wired or wireless connection port to a handheld, laptop or desktop computer. The connection port can also be used to communicate diagnostic information and previously stored performance information.

Outputs of the microcontroller, provided from the output buffers 426, are directed in part to the actuator 12 through a power driver circuit 410 and in part to the control panel 404. In one embodiment, the driver circuit converts the outputs to high voltage phases to drive an electrostatic actuator. The power driver circuit includes transformers and rectifiers to step up a-c waveforms generated by the microcontroller. In instances where the actuator is a DC motor, servomotor, or gear motor, the power driver circuit may be designed to generate high-current multi-phase signals.

When the operation mode of the muscle assistance device is set to apply a force that opposes the motion of the joint, the energy input from that ‘external’ force must be absorbed by the control circuit. While this energy can be dissipated as heat in a resistive element, it may also be returned to the battery in the actuator power supply 408 via a regeneration braking circuit 412. This concept is similar to “regenerative braking” found in some types of electric and hybrid vehicles to extend the operation time before the battery needs to be recharged.

In some embodiments, the microcontroller 402 can receive digital information via a digital interface connection 430 from a muscle stress sensor 416 that includes an analog to digital converter. In other embodiments the analog to digital converter can be located in the microcontroller 402 and the muscle stress sensor 416 can output analog data. The joint angle sensor 414 provides the joint angle through a variable capacitor which may be implemented as part of an electrostatic actuator. Alternatively, joint angle can be supplied by a potentiometer or optical sensor of a type known in the art, or by an encoder cupled to a lead screw or other drive component.

When the orthotic device is used to assist leg extension, the muscle stress sensor 416 may be implemented as a foot-pressure sensor wired to the active orthosis. In one embodiment, this sensor is implemented with parallel plates separated by a dielectric that changes total capacitance under pressure. The foot sensor may be a plastic sheet with conductive plates on both sides so that when pressure is applied on the knee the dielectric between the plates compresses. The change in the dielectric changes the capacitance and that capacitance change can be signaled to the microcomputer indicating to it how much pressure there is on the foot. There are pressure sensors that use resistive ink that changes resistance when pressure is applied on it. Other types of pressure sensors, such as strain gauges can be alternatively used to supply the pressure information. Further sensor constructs are subsequently described in more detail. These sensors are configured to detect the need or intention to exert a muscle. For example, the foot pressure sensor in conjunction with joint angle sensor detects the need to exert the quadriceps to keep the knee from buckling. Other types of sensors, such as strain gauges, can detect the intention by measuring the expansion of the leg circumference near the quadriceps. In another embodiment, surface mounted electrodes and signal processing electronics measure the myoelectric signals controlling the quadriceps muscle. When the orthotic device is used for other muscle groups in the body, appropriate sensors are used to detect either the need or intention to flex or extend the joint being assisted. It is noted that there may be a certain threshold (minimum amount of force), say 5 pounds on the foot, above which movement of the actuator is triggered.

Power for the muscle assistance device comes from one or more battery sources feeding power regulation circuits. The power for the logic and electronics is derived from the primary battery (in the power supply 408). The battery-charge state is fed to the microcontroller for battery charge status display or for activating low battery alarms. Such alarms can be audible, visible, or a vibration mode of the actuator itself. Alternatively, a separate battery can power the electronics portion.

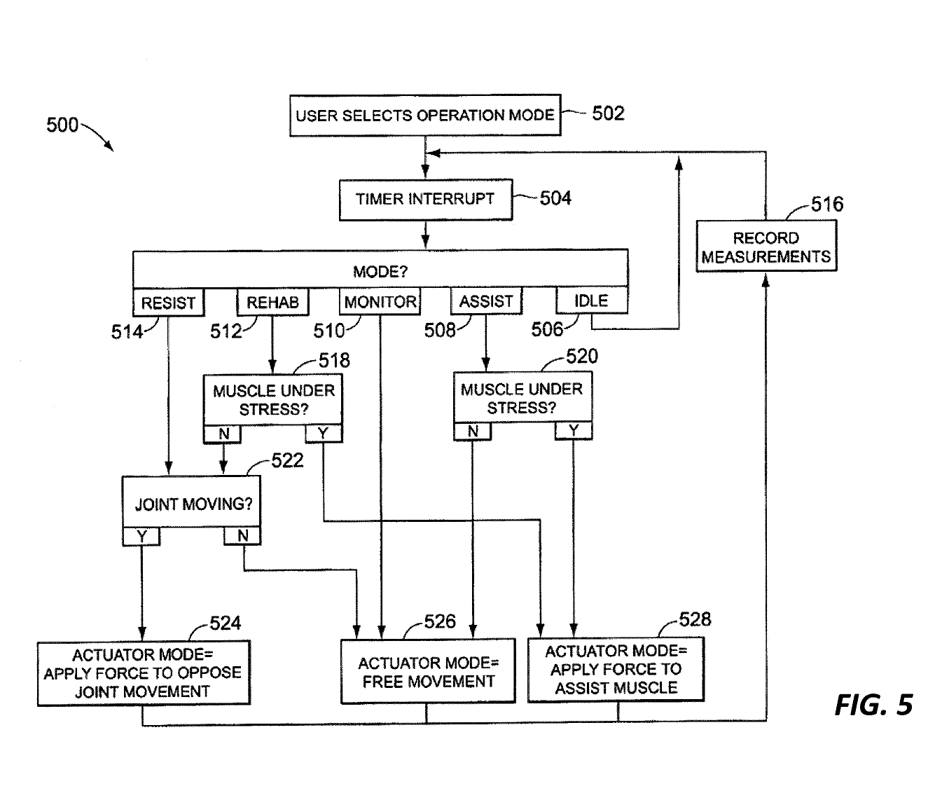

Turning now to FIG. 5, the operation of an exemplary muscle assistance device is illustrated with a block diagram. The algorithm in this diagram is implemented by embedded program code executing in the microcontroller. In the first step of FIG. 5, the user selects a mode of operation 502. The modes include: idle 506, assist 508, monitor 510, rehabilitate 512, and resist 514.

In the idle mode 506, the actuator is set to neither impede nor assist movement of the joint. This is a key mode in some implementations because it allows the device to move freely or remain in place when the user does not require assistance or resistance, or if battery has been drained to the point where the device can no longer operate. In idle mode, the actuator allows free movement with a clutch or an inherent free movement mode of the actuator, for example, even when primary power is not available.

In the monitor mode 510, the actuator is in free movement mode (not driven), but the electronics are activated to record information for later analysis. Measured parameters include a sampling of inputs from the sensors and counts of movement repetitions in each activation mode. This data may be used later by physical therapists or physicians to monitor and alter rehabilitation programs.

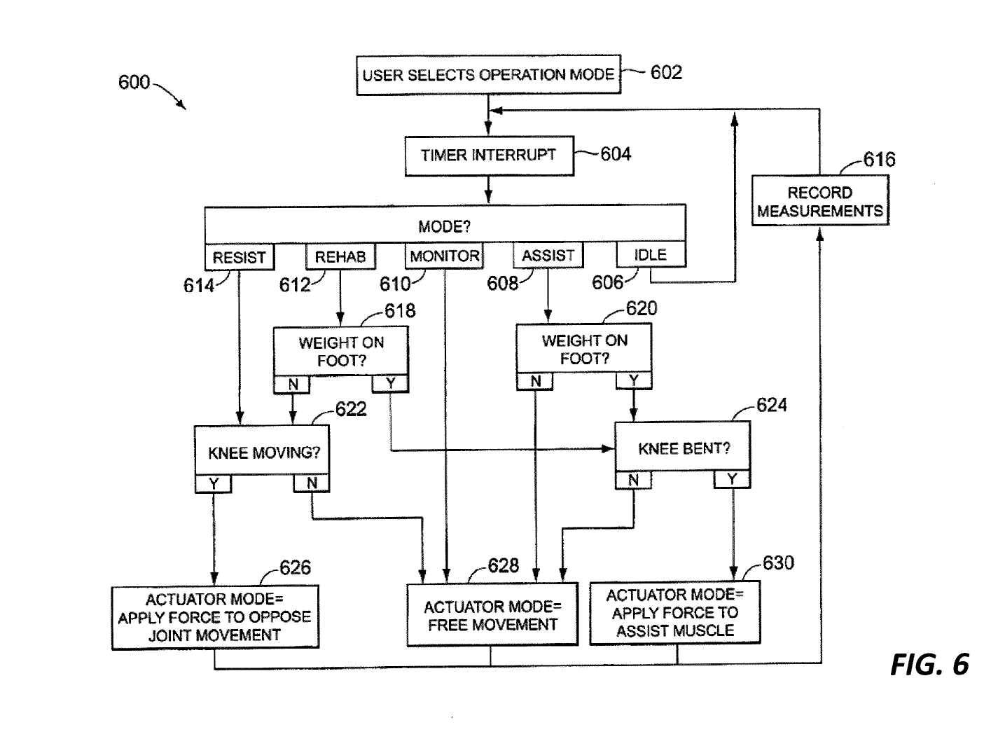

In the assist mode 508, the actuator is programmed to assist movements initiated by the muscle. This mode augments the muscle, supplying extra strength and stamina to the user. In the assist mode 508, the device can also resist the force exerted by gravity. This use of the term “resist” is not to be confused with the way the term “resist” is used in the description of the resist mode 514, as described below. Again, as mentioned herein with respect to FIGS. 5 and 6, “resist” can refer to both resisting gravity as described in the assist mode and to resisting the force exerted by muscle as described below in the resist mode.

In the resist mode 514, the device is operating as an exercise device. Any attempted movement is resisted by the actuator. Resistance intensity controls on the control panel determine the amount of added resistance. In the resist mode 514, the device resists the force exerted by the muscle.

In the rehabilitate mode 512, the device provides a combination of assistance and resistance in order to speed recovery or muscle strength while minimizing the chance of injury. Assistance is provided whenever the joint is under severe external stress, and resistance is provided whenever there is movement while the muscle is under little stress. This mode levels out the muscle usage by reducing the maximum muscle force and increasing the minimum muscle force while moving. The average can be set to give a net increase in muscle exertion to promote strength training. A front panel control provides the means for setting the amplitude of the assistance and resistance.

Then, assuming that the rehabilitate mode 510 is selected, a determination is made as to whether the muscle is under stress. The indicia of a muscle under stress is provided as the output of the muscle stress sensor reaching a predetermined minimum threshold. That threshold is set by the microcontroller in response to front panel functions.

If the muscle is not under stress or if the resist mode 514 is selected, a further determination is made as to whether the joint is moving 522. The output of the joint position sensor, together with its previous values, indicates whether the joint is currently in motion. If it is, and the mode is either rehabilitate or resist, the actuator is driven to apply force opposing the joint movement 524. The amount of resistance is set by the microcontroller in response to front panel settings. The resistance may be non-uniform with respect to joint position. The resistance may be customized to provide optimal training for a particular individual or for a class of rehabilitation.

If the joint is not in motion 522 or the monitor mode 510 is selected, the actuator is de-energized to allow free movement of the joint 526. This may be accomplished by using an actuator that has an unpowered clutch mode.

Additionally, if the muscle is under stress 520 or 522 and either the rehabilitate or the assist modes are selected, the actuator is energized to apply force for assisting the muscle 528. The actuator force directed to reduce the muscle stress. The amount of assistance may depend on the amount of muscle stress, the joint angle, and the front panel input from the user. Typically, when there is stress on the muscle and the joint is flexed at a sharp angle, the largest assistance is required. In the case of knee assistance, this situation would be encountered when rising from a chair or other stressful activities.

As mentioned before, when the device is in monitor mode 510, measurements are recorded to a non-volatile memory such as the flash memory of the microcontroller (item 420 in FIG. 4). Measurements may include the state of all sensors, count of number of steps, time of each use, user panel settings, and battery condition. This and the step of uploading and analyzing the stored information are not shown in the diagram.

FIG. 6 is a flow diagram specific to an active knee assistance device. This diagram assumes a specific type of muscle stress sensor that measures the weight on the foot. Relative to the diagram of FIG. 5, this diagram also shows a step (620) to determine whether the knee is bent or straight (within some variation). If the knee is straight, no bending force is needed 624 and power can be saved by putting the actuator in free-movement mode 630. To prevent problems such as buckling of the knee, the transitions, i.e., de-energizing the actuator, in both FIGS. 5 and 6 may be dampened to assure that they are smooth and continuous.

### Software

The software running on the microcontroller may be architected in many different ways. One architecture is to structure the embedded program code into subroutines or modules that communicate with each other and receive external interrupts (see item 424 in FIG. 4). Other embodiments are not interrupt driven. In one implementation the primary modules include control panel, data acquisition, supervisor, actuator control, and monitor modules. A brief description of these modules is outlined below.

The control panel responds to changes in switch settings or remote communications to change the mode of operation. Settings may be saved in a nonvolatile memory, such as a bank of flash memory.

The data acquisition module reads the sensors and processes data into a format useful to the supervisor. For instance, reading position from a capacitive position sensor involves reading the current voltage, driving a new voltage through a resistance, then determining the RC time constant by reading back the capacitor voltage at a later time.

The supervisor module may be a state machine for keeping track of high-level mode of operation, joint angle, and movement direction. States are changed based on user input and sensor position information. The desired torque, direction and speed to the actuator control the functioning of this module. The supervisor module may also include training, assistance, or rehabilitation profiles customized to the individual.

The actuator control module is operative to control the actuator (low level control) and includes a control loop to read fine position of the actuator and then drive phases to move the actuator in the desired direction with requested speed and torque. The monitor module monitors the battery voltage and other parameters such as position, repetition rates, and sensor values. It also logs parameters for later analysis and generates alarms for parameters out of range. This module uses the front panel or vibration of the actuator to warn of low voltage from the battery.

A number of variations in the above described system and method include, for example, variations in the power sources, microcontroller functionality and the like. Specifically, power sources such as supercapacitors, organic batteries, disposable batteries and different types of rechargeable batteries can be used in place of a regular rechargeable battery. Moreover, microcontroller functionality can be split among several processors or a different mix of internal and external functions. Also, different types of orthotic devices, with or without hinges and support frames, may be used for attachment to the body, and they may be of different lengths. Various ways of communicating the ‘weight-on-foot’ may be used, either through wired or wireless connections to the control circuitry, or by making the orthosis long enough to reach the foot.

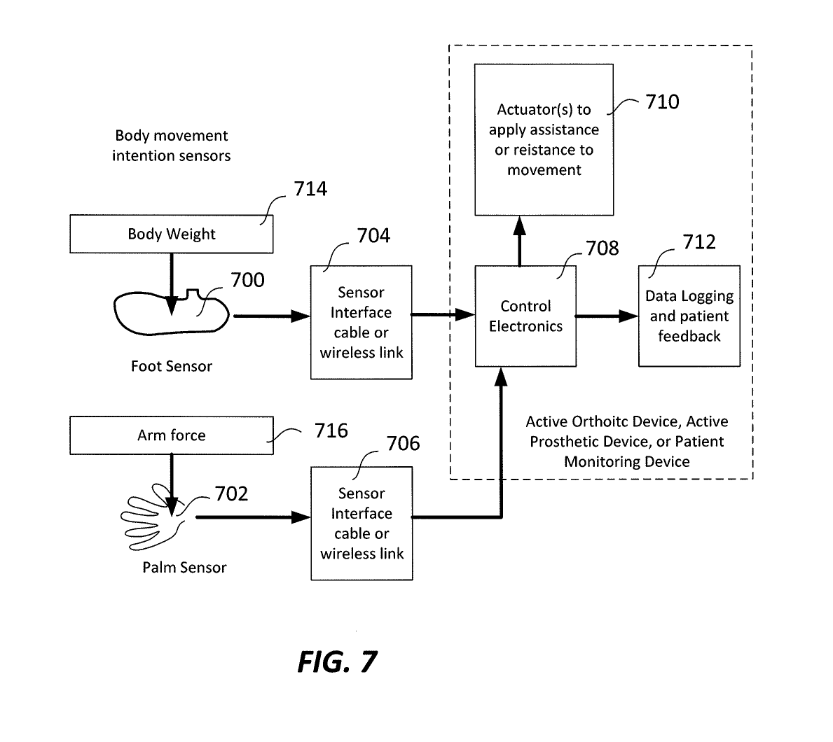

FIG. 7 is a block diagram illustrating an embodiment of a sensor for use in an orthotic device. Examples of orthotic devices and orthotic device sensors are discussed above and are also disclosed in U.S. Pat. Nos. 6,966,882 and 7,239,065, and U.S. application Ser. No. 12/703,067, which are hereby incorporated by reference in their entireties. In some embodiments, a foot sensor 700 can be used to determine the intention (or residual intention after a stroke) of a patient to move or use his leg. For example, the foot sensor 700 can have separate heel and ball portions to measure the distribution of the weight 714 of the patient on the foot in order to determine the required force and timing for augmenting the force of the quadriceps and other leg muscles using the active orthotic device during different activities such as stair climbing, walking, and rising up or sitting down, for example. By using the active orthotic device to augment the residual intention of a stroke patient, neuroplastic recovery can be promoted.

In some embodiments, a palm sensor 702 can be used to detect the force 716 exerted on or by the arm for controlling an active orthotic device to help the patient use an object, such as the arms of a chair or a handrail for example, to stand or balance or to partially support the body weight of the patient through a cane or walker held by the paretic hand. Normally a hemiparetic stroke patient is unable to hold a cane on the paretic side, and holding the cane on the unaffected side causes weight to be shifted to the unaffected side, which can result in a pathological gait over time, and additionally can lead to an increased chance of falls. The devices and methods disclosed herein can help overcome these issues.

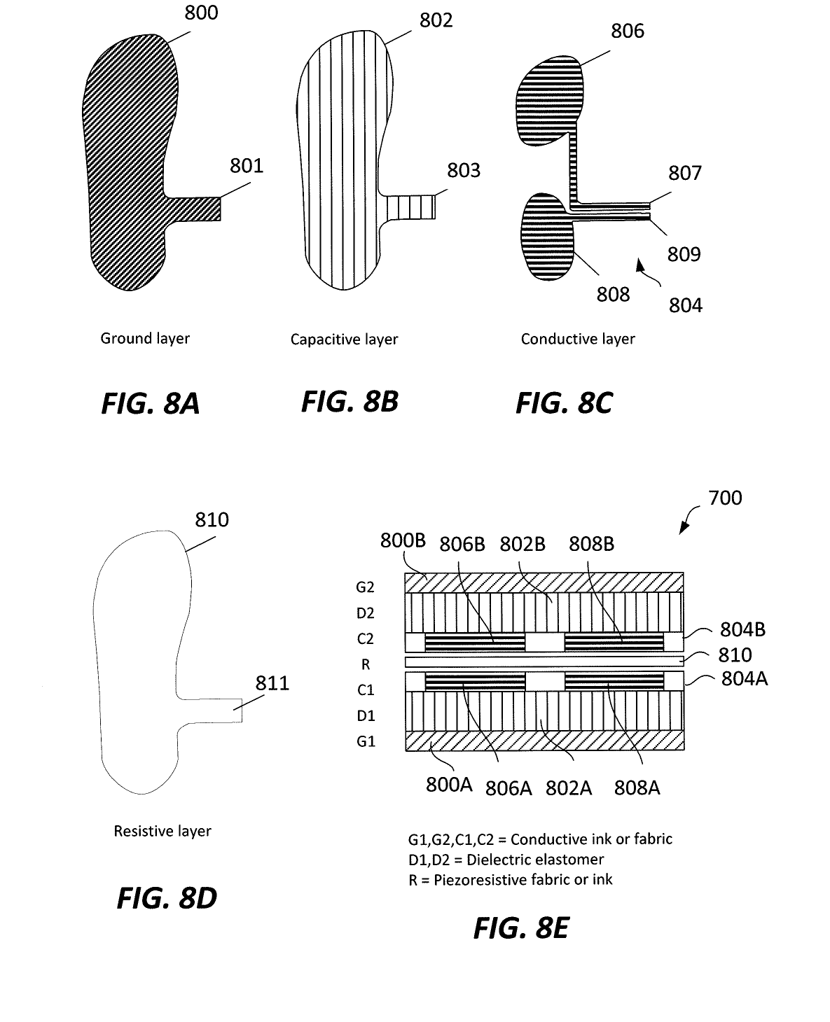

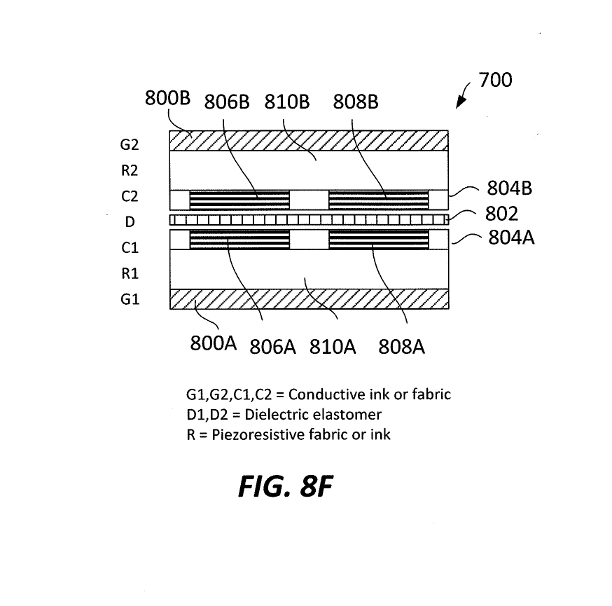

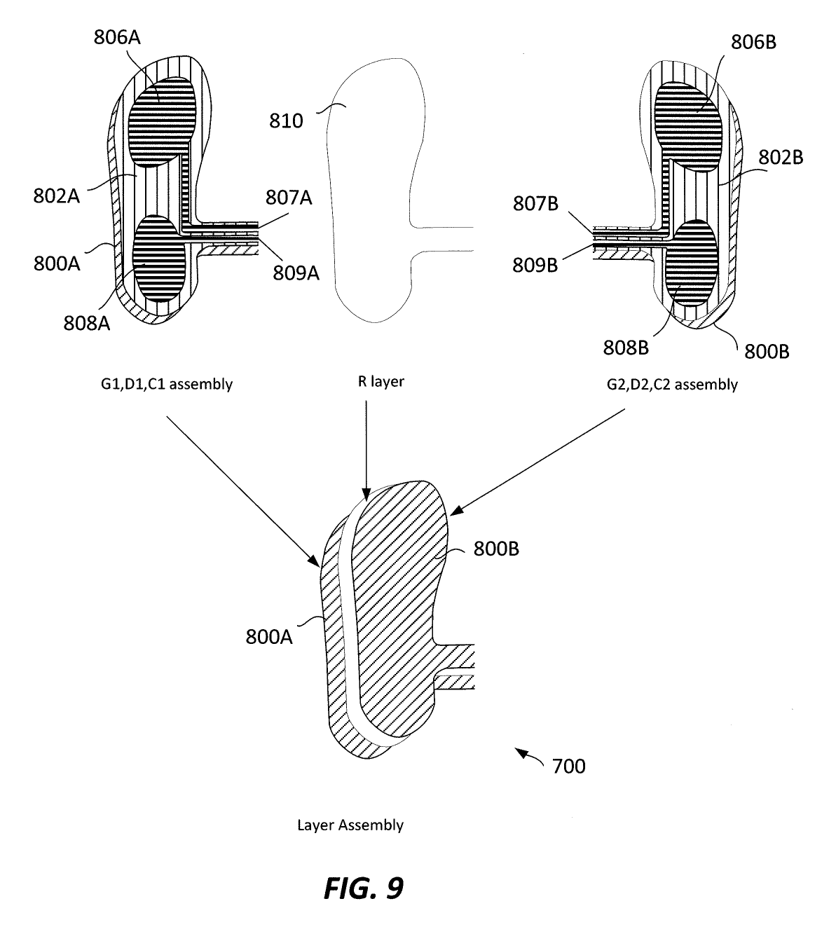

FIGS. 8A-8F illustrate the plurality of layers that can be used to form an embodiment of a foot sensor that provides foot sensing information as well as fault detection and fault tolerance. In FIGS. 8 and 9, the foot-shaped portions are positioned under the foot and the narrow tab portions 801, 803, 807, 809, 811 that extend from the foot-shaped portions are bent up to exit the shoe and make the connection to the control electronics. The sensing technology described herein can be used to replace other types of force sensors, e.g. load cells, at a lower cost. FIG. 8A illustrates an embodiment of a ground layer 800. The ground layer 800 is a conductive layer that can form the outer layers of the sensor. The ground layer 800 can be formed from a variety of conductive materials, such as a conductive ink like a silver based ink from Creative Materials, a conductive ink with graphene conducting elements such as Vor-Ink™ from Vortex Materials, a conductive fabric such as a silver conductive fabric from Marktek Inc. such as SBA1317 or CN-4190 nickel on copper-plated polyester fabric tape from 3M, or any other suitable conductive fabric or polymer. Layers, such as the ground layer 800, can be formed by printing the flexible conductive ink onto a substrate, which can be another sensor layer, such as the dielectric layer of a capacitive sensor or the piezoresistive layer of a resistive sensor. The conducting layers may be printed with gaps or as stripes rather than as continuous filled regions, thereby reducing the total amount of conductive ink required. Reducing the amount of ink is particularly advantageous when using an expensive ink such as one based on silver. Alternatively, the conductive layers can be made by bonding, attaching or adhering a conductive fabric to the substrate as describe herein. Silver based conductive materials can have antibacterial and/or antimicrobial properties and can be used in any patient facing layer, or any other layer requiring a conductive material. Other antibacterial and/or antimicrobial agents or materials, such as copper or zinc based compounds or alloys, can be used in place of silver to give the layers antibacterial properties. The ground layer 800 and the conductive materials used to form the ground layer can be flexible. The ground layer 800 can be generally foot shaped to match the contour of the patient's foot. Extending from the foot shaped portion of the ground layer 800 is a ground layer connector 801 that forms a sensor connector when combined with the other sensor layer connectors described herein.

FIG. 8B illustrates an embodiment of a capacitive layer 802. The capacitive layer 802 can be made from, for example, a dielectric material that has a variable capacitance depending on the level of compression of the dielectric material or the level of force exerted on the dielectric material. For example, the dielectric material can be made from a reversibly compressible insulator such as microcellular urethane, for example provided by Rogers Corporation as Poron™, or any other suitable reversibly compressible foam or porous polymer or material. The capacitance measured by a capacitive sensor incorporating the capacitive layer 802 increases as force is applied and the capacitive layer 802 is compressed. This relationship allows the force exerted on the capacitive layer 802 to be determined by measuring the capacitance. The capacitive layer 802 can be generally foot shaped to match the contour of the patient's foot.

FIG. 8C illustrates an embodiment of a conductive layer 804 having a ball portion 806 to form a ball sensor and a heel portion 808 to form a heel sensor. The ball portion 806 can be shaped generally like the ball of the patient's foot, and the heel portion 808 can be shaped generally like the heel of the patient's foot. In some embodiments, the ball portion 806 and/or the heel portion 808 can be further subdivided into a plurality of portions to increase the resolution of the distribution of weight from the patient's foot. In other embodiments, the conductive layer 804 can be formed as a single layer or portion that can be generally foot shaped to match the contour of the patient's foot. The conductive layer 804 can be formed from a variety of conductive materials, such as the materials described above for the ground layer 800, including for example, conductive ink or conductive fabric. Extending from the ball portion 806 of the conductive layer 804 is a ball portion connector 807 and extending from the heel portion 808 is a heel portion connector 809 that form a sensor connector when combined with the other sensor layer connectors described herein. The ball portion connector 807 and the heel portion connector 809 are collectively called conductive layer connectors 807, 809. As shown in FIGS. 8E and 9, the assembled sensor includes two conductive layers 804A, 804B, each comprising a ball portion 806A, 806B and a heel portion 808A, 808B with conductive layer connectors 807A, 807B, 809A, 809B.

FIG. 8D illustrates an embodiment of a resistive layer 810. The resistive layer 810 can be made from a variety of resistive materials that have a variable resistance depending on the amount of mechanical force applied to the surface of the material. This relationship allows the force exerted on the resistive layer 810 to be determined by measuring the resistance. For example, a piezoresistive material like EeonTex™ NW-170-SL-PA-1700 provided by Eeonyx Corporation can be used to fabricate the resistive layer. The resistive layer 810 can be generally foot shaped to match the contour of the patient's foot, with separate independent sensors formed wherever there is a conductive material above and below the resistive material.

The plurality of layers that can be used to form an embodiment of the sensor can be made of flexible fabrics or other flexible materials to form a flexible sensor and can be used, for example, as a shoe insert, sewn to a sock or slipper, built into a shoe, a glove insert, sewn to a glove, or attached to an orthotic device such as an ankle-foot-orthotic device.

FIG. 8E is a cross-sectional view of the plurality of sensor layers after assembly to form an embodiment of a foot sensor 700. In this embodiment, the ground layers 800A, 800B form the outer layers of the foot sensor 700. Moving inwards, two capacitive layers 802A, 802B are disposed adjacent to and in contact with the ground layers 800A, 800B. Two conductive layers 804A, 804B are disposed adjacent to and in contact with the capacitive layers 802A, 802B, such that a capacitive layer 802A, 802B is disposed between a conductive layer 804A, 804B and a ground layer 800A, 800B. The two conductive layers 804A, 804B have a ball portion 806A, 806B and a heel portion 808A, 808B that correspond to the ball and heel of a patient's foot. In the middle, a resistive layer 810 is disposed between and in contact with the two conductive layers 804A, 804B. This configuration is advantageous when the cost of the resistive layer is greater than the cost of the capacitive layer because only a single resistive layer is used while two capacitive layers are used, and therefore, such a configuration reduces material costs. Another advantage provided by this configuration is that the two capacitive layers are better shielded and/or grounded, thereby reducing noise in the sensor system.

In some embodiments, as illustrated in FIG. 8F, the location of the resistive layer 810 can be swapped with the location of the capacitive layers 802A, 802B, which means the sensor has a single capacitive layer 802 disposed between the two conductive layers 804A, 804B, and two resistive layers 810A, 810B where each resistive layer is disposed between a conductive layer 804A, 804B and a ground layer 800A, 800B.

FIG. 9 illustrates the layer assembly and orientation of an embodiment of a foot sensor 700. A first subassembly of the foot sensor 700 can be assembled from a ground layer 800A, a capacitive layer 802A, a ball portion 806A of the conductive layer 804A, and a heel portion 808A of the conductive layer 804A. The capacitive layer 802A can be layered over the ground layer 800A, and the ball portion 806A and heel portion 808A of the conductive layer 804A can be layered over the capacitive layer 802A. A second subassembly of the foot sensor 700 can be assembled as the mirror image of the first subassembly of the foot sensor 700. The second subassembly has a ground layer 800B, a capacitive layer 802B layered over the ground layer 800B, and a ball portion 806B and a heel portion 808B of the conductive layer 804B layered over the capacitive layer 802B.

To assemble the foot sensor 700, the first subassembly and second subassembly are combined together with a resistive layer 810 placed in between the first subassembly and the second subassembly such that a first surface of the resistive layer 810 is adjacent to and contacts the conductive layer 804A of the first subassembly and the second surface of the resistive layer 810 is adjacent to and contacts the conductive layer 804B of the second subassembly, resulting in a layer orientation as described also with reference to FIG. 8E.

Although a foot sensor 700 has been illustrated in FIGS. 8A-8F and FIG. 9, a hand sensor 702 or other body part sensor can be formed in a similar manner as described above. For example, a hand or palm sensor 702 can be made of a plurality of sensor layers, including at least one hand shaped or palm shaped ground layer, at least one hand shaped or palm shaped capacitive layer, at least one hand shaped or palm shaped resistive layer and a conductive layer that can be hand shaped or palm shaped or formed from a plurality of different portions that correspond to different parts of the hand, such as a palm portion and digit portions. The sensor layers can be arranged as described above for the foot sensor 700. The descriptions in this application related to the foot sensor 700 are applicable and can be used with the hand sensor 702 or other body part sensor embodiments. For example, the integrated electronics described below for the foot sensor are applicable to the hand sensor 102 and other body part sensor.

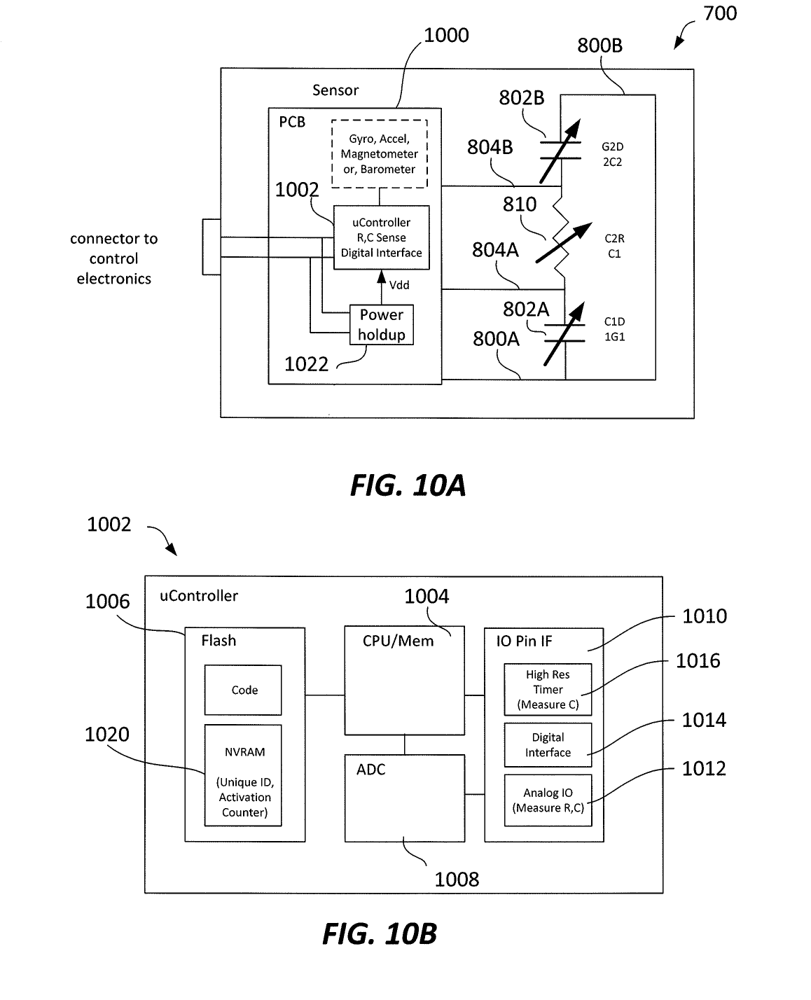

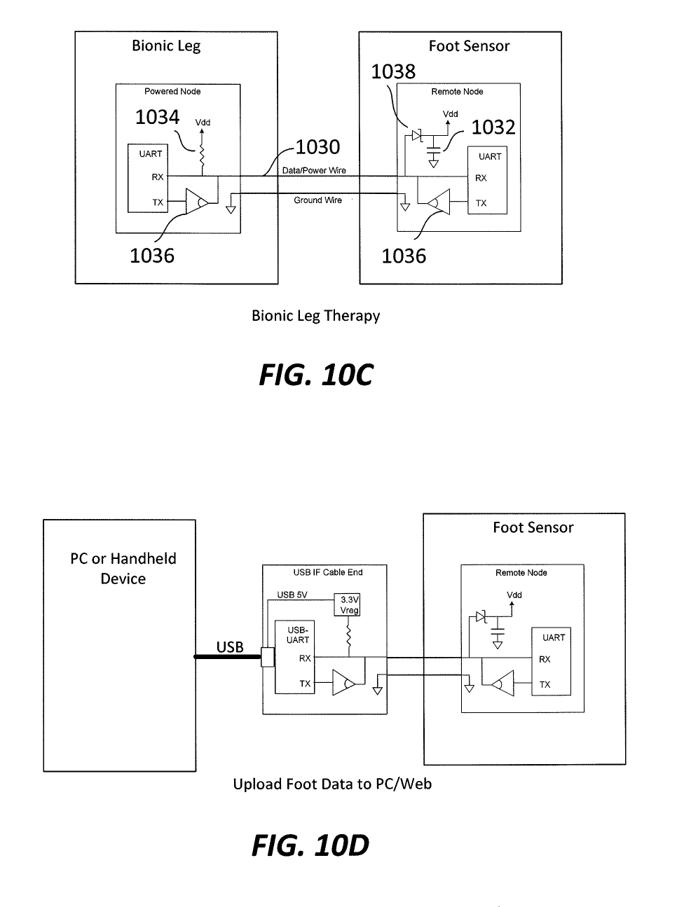

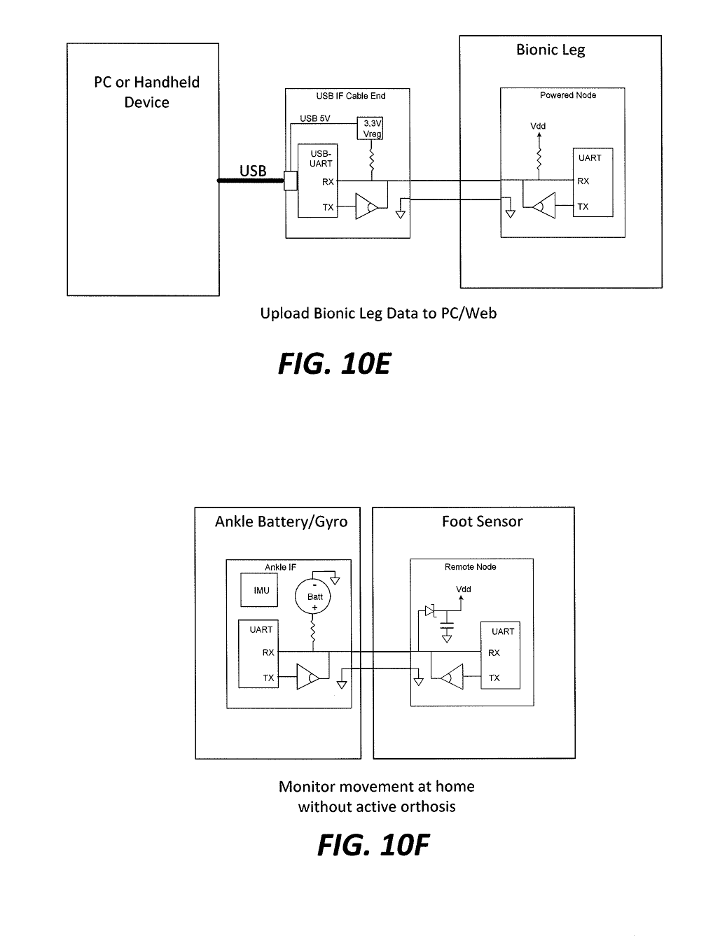

FIG. 10A is a block diagram illustrating an embodiment of a foot sensor 700 with integrated electronics 1000 to determine the capacitance of the capacitive subassemblies including the capacitive layers 802A, 802B and the resistance between the capacitive subassemblies separated by the resistive layer 810 and to communicate the data to a monitoring device and/or active orthotic device. The integrated electronics 1000 can be a printed circuit board (PCB) with a microcontroller 1002. The microcontroller 1002, for example a MSP430 microcontroller provided by Texas Instruments illustrated in FIG. 10B, can include a processor or processing unit 1004, memory 1006, an analog-to-digital converter (ADC) 1008, an input-output interface 1010 with an analog interface 1012 to measure capacitance and resistance, a digital interface 1014 with a ground wire and a single bidirectional data wire, such as a serial port (UART) with open-drain driver and pullup resistor to supply power (shown in FIG. 10C), and a high resolution timer 1016 for measuring capacitance. The Texas Instruments MSP430 family of microcontrollers is low cost, low power and includes capacitive sensing features. A suitable microcontroller from the MSP430 family is the MSP430G2112 in a 14-pin thin-shrink small outline package (TSSOP) with dimensions of 5 mm by 4.4 mm. The Microchip PIC12F is another suitable family with devices in 8, 14-pin and larger packages. Both the processing unit 1004 and the ADC 1008 can be operably connected to the input-output interface 1010. The processing unit 1004 can additionally be operably connected to the memory 1006 and the ADC 1008. In some embodiments, the PCB 1000 can include additional sensors including for example a gyroscope, an accelerometer, a barometer, a magnetometer and/or a global positioning system (GPS) device. The additional sensors can be operably connected to the processing unit 1004 on the microcontroller 1002.

As illustrated in FIGS. 7 and 10A-10F, the digital interface 1014 allows the PCB to communicate with control electronics 708 that can activate actuators 710 in an active orthotic or prosthetic device to apply assistance or resistance to movement, or send the data to a patient monitoring device 712, such as a PC, mobile device or handheld device for example, for data logging, data analysis and patient feedback. The digital interface 1014 can be operably connected to the control electronics 708 through any means, such as a direct connection via a wire or via a wireless connection between a transmitter and receiver. As described above, the digital interface 1014 can have a ground wire connection and a single bidirectional data wire that provides the ability to communicate data in both directions. As illustrated in FIGS. 10A-10C, the bidirectional data wire 1030 can also provide power to the PCB by charging a capacitor 1032 via diode 1038 in a power hold-up circuit 1022 during the time between data transmissions. Although the bidirectional data wire 1030 has been described using the term “wire,” it should be understood that the wire can be a conductive trace or conductive line or other suitable medium for data transmission.

The digital interface 1014 can use an open-drain pull-up resistor 1034 at one end of the bidirectional data wire 1030, open drain drivers 1036 at both ends, and a protocol to arbitrate and determine when a device at one end or the other is allowed to send data over the bidirectional data wire 1030. The drivers and receivers 1036 can be connected to universal asynchronous receivers/transmitters (UARTs) to covert parallel data to serial data.

The memory 1006 can be flash memory and can store programming and/or code and/or instructions, which when executed by the processing unit, causes the processing unit to perform a variety of functions described herein, such as, for example, measuring the resistance and capacitance of the sensor 700. Resistance can be measured by adding a fixed resistor of known resistance in series with the variable resistance of the resistive layer 810 and driving a voltage across the two resistive components. The ADC 1008 of the microcontroller 1002 can measure the voltage across the fixed resistor of known resistance and the variable resistance of the resistive layer 810, both the voltage drop in combination and the voltage drop across each individual component. The voltage drop across the fixed resistor of known resistance divided by the known resistance gives the current through both the fixed resistor and the variable resistance of the resistive layer 810. The resistance of the variable resistance of the resistive layer 810 can be determined by dividing the voltage across the variable resistance of the resistive layer 810 by the current.

Capacitance can be measured by either digitally counting the frequency of a relaxation oscillator, or with the ADC 1008 by measuring the time constant to charge or discharge the capacitor. Capacitive sensing capability is included in commercially available microcontrollers such as the Texas Instruments MSP420 microcontroller and the Microchip PIC12F series of microcontrollers. Both these methods are described in more detail in Zack Albus, PCB-Based Capacitive Touch Sensing With MSP430, Texas Instruments Application Report SLAA363A—June 2007—Revised October 2007, which is herein incorporated by reference in its entirety.

Note that capacitive layers 802A and 802B serve a dual purpose. In the areas in the ball and heel regions, the capacitive layer is the dielectric of the capacitors that change value as force is applied, while capacitive layer portion not under the ball and heel are used only as an insulator to prevent shorting out between the ground layer 800A and conductive layer 804A, or between ground layer 800B and conductive layer 804B.

In areas where only insulation is required, another suitable insulator could be substituted for the insulation provided by the capacitive or resistive layers. In some embodiments, an insulating ink is applied to cover the area where the leads connect to the sensing areas. This may be advantageous because it reduces the required amount of resistive material, and it prevents or reduces inaccuracies that could be introduced by unintentional compression of the lead-connection areas.

In addition, the programming and/or code and/or instructions, which when executed by the processing unit, may be configured to cause the processing unit to determine whether a portion of the sensor 700 is faulty, and may continue operation of the sensor 700 in a predetermined manner that depends of which portion of the sensor is faulty 700, as will be described in further detail below. In addition, the memory 1006 can store additional data 1020 including a unique identification, which can be a unique serial number or a unique patient identification, for example, and can also store an activation count and/or a step count which can be used to determine the sensor end of life, as discussed further below.

The memory can also store patient-specific usage information downloaded by the controller. At the beginning of a series of therapy sessions, a footpad may be assigned to a patient. As the therapy progresses, information related to the quantity and quality of movement can be downloaded to memory in the foot sensor. At the end of the therapy or at some pre-defined interval, the sensor can be returned to a facility that reads the data and produces a patient report. Alternatively, data from the sensor can be wirelessly uploaded for later use reporting progress. In addition, as illustrated in FIGS. 10D and 10E, data from the foot sensor and orthotic device can be uploaded to a PC, mobile device, or other handheld device, which can then transmit the data through the internet or a data network to a server or other processing device for further analysis and/or storage. The foot sensor and orthotic device can be connected to the computing device via a wired connection or a wireless connection. For example, the wired connection can be accomplished using a serial data connection, such as a USB connection. A USB interface cable can be provided with both a USB connector to connect to the computing device on one end, and a connector for interfacing with the foot sensor or orthotic device on the other end. The connector for interfacing with the foot sensor or orthotic device can be a self-aligning magnetic connector as further described below.

FIG. 10F illustrates another embodiment of the foot sensor, where the foot sensor can be used without an active orthosis. Instead, the foot sensor can be connected, wired as illustrated or wirelessly in other embodiments, to an ankle device which may include other sensors such as, for instance, an inertial measurement unit, which uses gyroscopes and accelerometers to measure movement, position and orientation of the ankle and foot. Other potential sensors include a magnetometer, barometer, temperature sensor or GPS sensor. The ankle device can include a battery to provide power to the ankle device and the foot sensor when disconnected from a main power source. This set up allows data to be captured with just the foot sensor and the relatively small ankle device, thereby allowing the patient and health care provider to monitor the patient's movement characteristics at home without needing an active orthosis. This data can be used to monitor the patient's recovery progress and can be used to customize and/or tailor the parameters of an active orthosis for use in rehabilitating the patient.

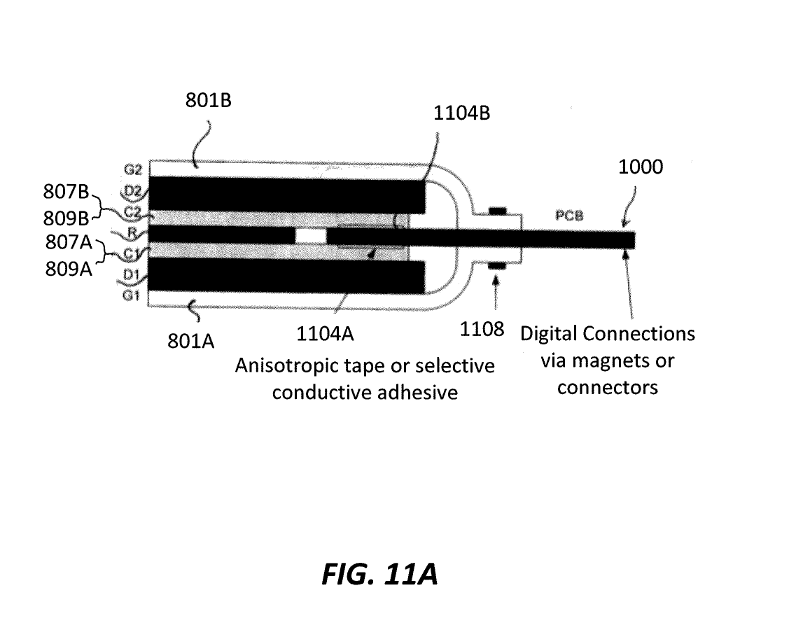

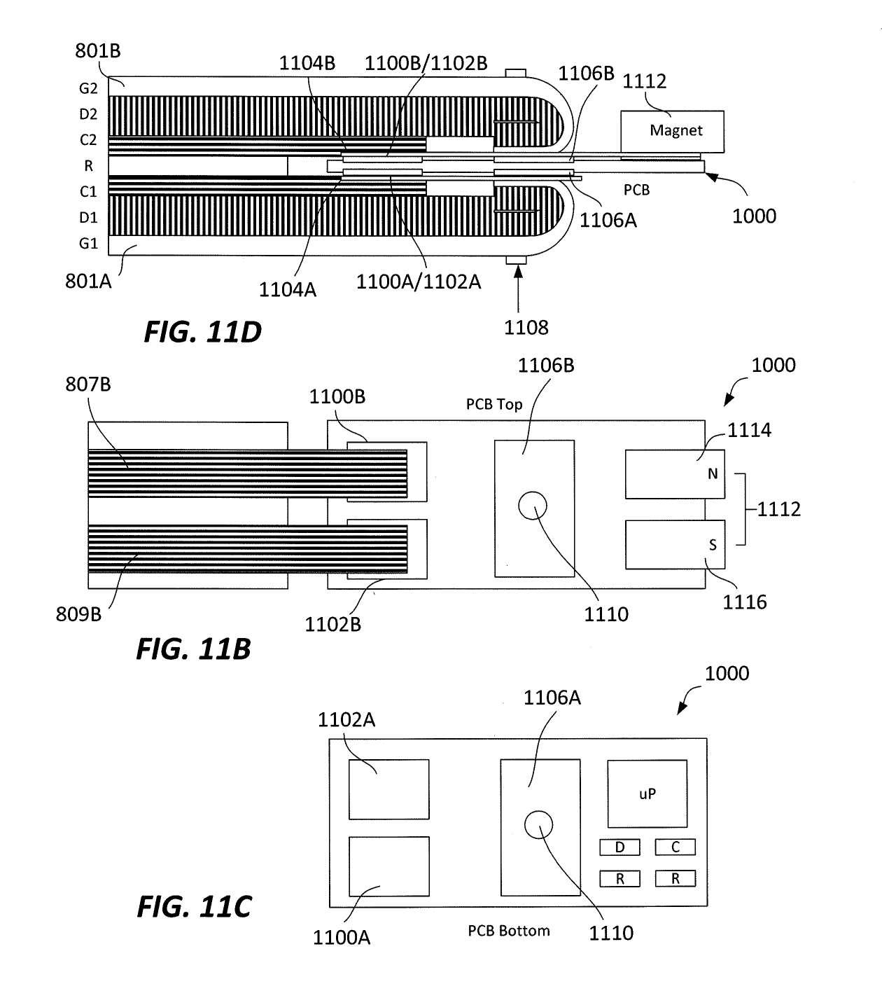

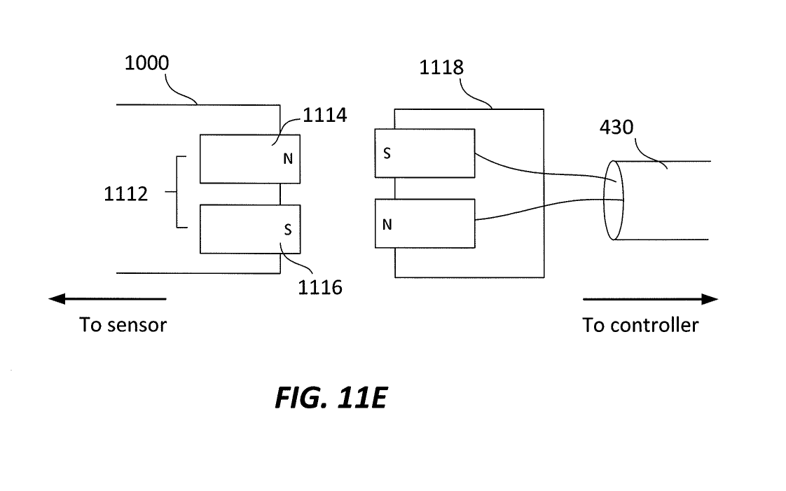

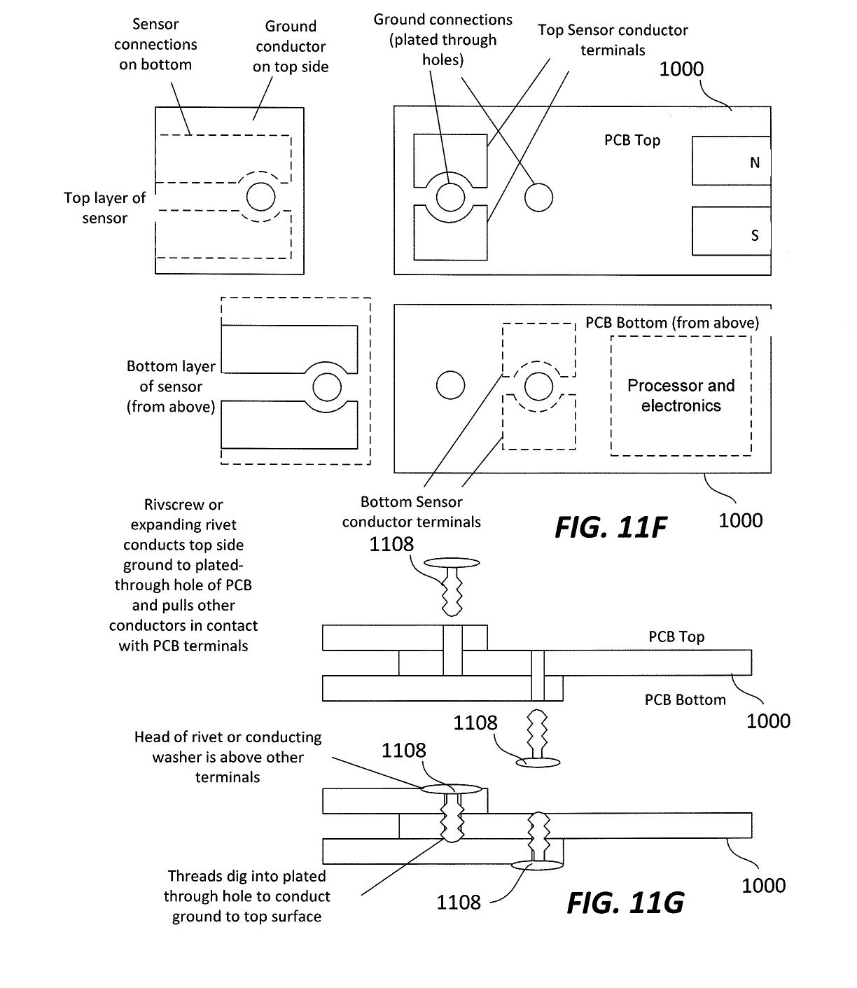

FIGS. 11A-11F illustrate how the PCB 1000 may be connected with the sensor layer connectors. FIG. 11A illustrates in one embodiment a cross-sectional view of the sensing layer connectors in connection with a PCB 1000. FIG. 11B illustrates a top view of PCB in connection with two conductive layer connectors 807B, 809B. FIG. 11C illustrates a bottom view of the PCB 1000. FIG. 11D illustrates in another embodiment a cross-sectional view of the sensing layer connectors in connection with a PCB 1000. The ends of the conductive layer connectors 807A, 809A, 807B, 809B can be connected to the corresponding conductive connector contacts 1100A, 1102A, 1100B, 1102B on the PCB 1000 using, for example, conductive tape 1104A, 1104B, conductive adhesive or some other suitable conductive material. The conductive tape 1104A, 1104B can be anisotropic, z-axis conductive tape, such as 3M 9703 conductive tape or the equivalent. In some embodiments, conductive tape 1104A, 1104B or conductive adhesive can also be used to connect, fasten or secure the ground layer connectors 801A, 801B to the corresponding ground layer contacts 1106A, 1106B on the PCB 1000. Other mechanical means, eg. screws, rivets or latches may also be added to securely attach the PC board to the sensor. By locating the processor or processing unit on a PCB 1000 in close proximity to the sensors, stray capacitance and RF interference can be minimized or reduced for improved sensor accuracy and precision.

As shown in FIGS. 11A and 11D, in some embodiments the ends of the ground layer connectors 801A, 801B can be connected or fastened to the corresponding ground layer contacts 1106A, 1106B on the PCB 1000 using a rivet 1108. A hole 1110 or via can be formed in the ground layer contacts 1106A, 1106B and through the PCB 1000 to receive the rivet 1108. Holes can also be formed in the end portions of the ground layer connectors 801A, 801B to receive the rivet 1108. In addition, in some embodiments, holes for receiving the rivet 1108 can be formed in the end portion of the capacitive layers 802A, 802B. In some embodiments, the rivet 1108 can be made from an electrically conductive material, such as a metal, and can function additionally to electrically couple the two ground layer connectors 801A, 801B together and to a plated-through ground connector hole in the PC board. In other embodiments, the ground layer connectors 801A, 801B can be additionally or alternatively fastened or connected to the ground layer contacts 1106A, 1106B using a conductive tape or a conductive adhesive.

In some embodiments as shown in FIG. 11D, the rivet 1108 can fasten multiple layers to the PCB, such as the ground layer connectors 801A, 801B. In some embodiments, the ground layer connectors 801A, 801B can be made to contact the ground layer contacts 1106A, 1106B by bending or folding the end of the sensor layer connector over on itself so that the rivet 1108 contacts one portion of the ground layer connectors 801A, 801B and the ground layer contacts 1106A, 1106B contacts another portion of the ground layer connectors 801A, 801B, with another sensor layer, such as the capacitive layer 1104A, 1104B, folded in between. The connection shown in FIG. 11A may be advantageous when the outer conducting layers, such as the ground layer connectors 801A and 801B, are separable from the underlying layer, such as when the outer conducters are made from a conducting fabric, while the connection shown in FIG. 11D may be advantageous where the conducting layer is not separable from the underlying layer, such as when the conducting layer is made from a conductive ink. In some embodiments, rivet 1108 is conducting and it provides the connection between ground layer connections 801A and 801B, and also provides a connection to the ground of the PCB via a press-fit connection to the plated through hole on the PCB.

As illustrated in FIG. 11B, the PCB 1000 can have conductive magnets 1112, including a magnet with an external north pole 1114 and a magnet with an external south pole 1116, attached to the PCB 1000 pads by conductive epoxy, conductive tape, conductive adhesive, or some other suitable conductive material. Each of the conductive magnets 1112 is operably connected or electrically connected to one of the ground wire or the bidirectional data wire. For example, the external north pole 1114 can be operably connected to the ground wire and the external south pole 1116 can be connected to the bidirectional data wire, or vice versa.

As illustrated in FIG. 11E, a magnetic connector 1118 can be used to releasably connect a device such as a controller to the conductive magnets 1112 on the PCB 1000. The mating magnetic connector 1118 is wired to the controller and includes a ground wire connector and a bidirectional wire connector with magnets having poles reversed from the polarity of the conductive magnets 1112 on the PCB 1000, where the magnets can also be conductive. For example, if the external north pole 1114 is connected to the ground wire and the external south pole 1116 is connected to bidirectional data wire, the magnetic connector 1118 will have a ground wire connector with a magnet having an external south pole and a bidirectional data wire connector with a magnet having an external north pole. This arrangement results in a self-aligning magnetic connector 1118 that is releasably attached to the conductive magnets 1112 on the PCB 1000. As the magnetic connector 1118 comes near the conductive magnets, the magnetic connector 1118 automatically snaps into place over the conductive magnets 1112 with the correct polarity, meaning the connection cannot be made in reverse due to the repulsive force of the magnets when the orientation is improper. If a removal force exceeding a predetermined threshold force is exerted on the magnetic connector 1118 after connection with the conductive magnets 1112, the magnetic connector 1118 will reversibly detach from the conductive magnets rather than break the PCB or sensor assembly. The predetermined threshold force for detachment can be adjusted by varying the strength of the magnets in the magnetic connector 1118 and/or the conductive magnets 1112. For example, a magnet with a predetermined magnetic strength can be selected for a desired predetermined threshold force for detachment.

FIGS. 11F and 11G illustrate another embodiment of the connection between the PCB 1000 and sensor. The PCB 1000 can have top sensor conductor terminals on the top surface of the PCB 1000 and bottom sensor conductor terminals on the PCB 1000 bottom. A ground connection, which can be a plated through hole such as a via, can be provided on the PCB 1000 at each set of conductor terminals. In some embodiments, the conductor terminal sets can be offset from each other, when, for example, the connector, such as a rivet, does not extend all the way through the PCB and sensor. In other embodiments, the sensor conductor terminals can be symmetrically located on opposing sides of the PCB, when, for example, the connector, such as a rivet, extends all the way through both the PCB and sensor.

The rivet 1108 can be, for example, a Rivscrew® brand expanding rivet that conducts the top side ground to the plated-through hole of the PCB and pulls the other conductors in contact with the PCB terminals. The head of the rivet and/or an added washer can be used to compress the conductors to the PCB terminals and ensure an adequate electrical contact between the parts. In addition, the rivet can expand, which enhances the contact of the rivet threads with the plated through hole to conduct ground to the top and/or bottom surfaces.

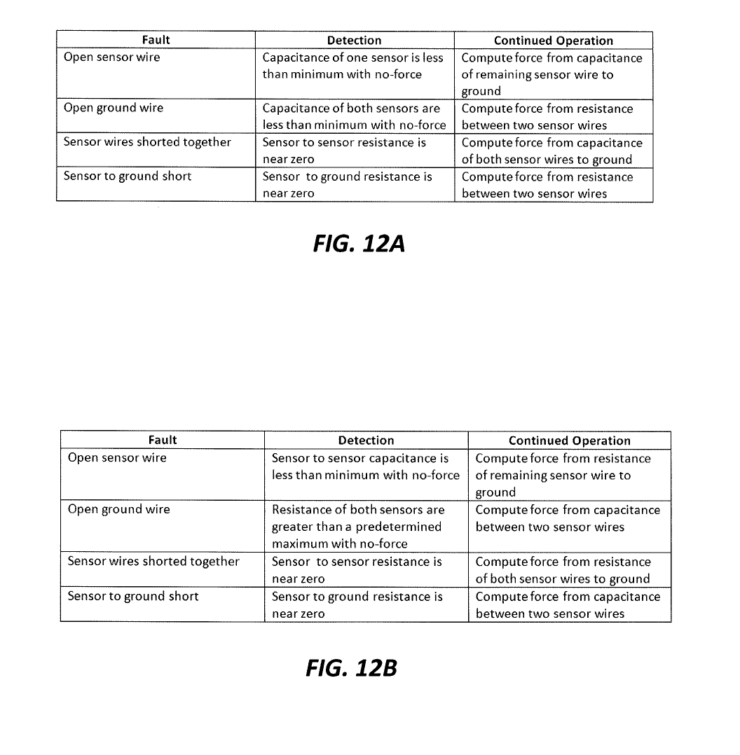

FIG. 12A illustrates an embodiment of the fault detection capabilities built into the sensor and how the sensor can continue to operate despite the presence of one or more faults in a sensor having a sensor layer configuration shown in FIG. 8E. For illustrative purposes, FIGS. 12A and 12B will be described with respect to the ball portion 806A, 806B on the assembled sensor. This description is also applicable to the heel portion 808A, 808B or any other sensor assembled in a manner described herein. One possible fault is an open sensor wire/conductive layer connector 806A, 806B, where open sensor wire can refer to a break or disruption in one of the wires/conductive layer connectors 806A, 806B that is connected to one of the conductive layers 804A, 804B shown in FIG. 8E. For example, a break or disruption of one of the conductive layer connectors 807A, 807B can result in an open sensor wire fault. The open sensor wire fault can be detected by measuring the capacitance of the capacitive subassemblies which include capacitive layers 802A, 802B and determining whether the capacitance of one of the capacitive subassemblies is less than a predetermined minimum capacitance when no force is exerted on the sensor by the patient. The predetermined minimum capacitance can be determined based on the known properties of the dielectric material, through a calibration procedure performed in the factory, or based on a minimum capacitance value during patient use.

Continued operation of the sensor is possible by disregarding the capacitance measurements from the open sensor wire and measuring the capacitance of the capacitive subassembly with the functional conductive layer/sensor wire and ground layer/ground wire. For example, with reference to FIG. 8E, FIG. 10A and FIG. 12A, if the sensor wire/conductive connector 806A is open, the capacitance of the capacitive subassembly that includes capacitive layer 802A cannot be accurately determined. However, the sensor wire/conductive connector 806B is still functional, so the capacitance of the conductive subassembly that includes capacitive layer 802B can still be determined, which will allow the device to determine the force exerted on the sensor. In order to accurately measure the capacitance of a particular capacitive subassembly, the ground layer 800A, 800B and the ground wire has to be functional and not open, and the conductive layer 804A, 804B and the associated sensor wire/conductive connector 806A, 806B adjacent the particular capacitive layer 802A, 802B also has to be functional and not open. Generally, in order to measure the properties of a particular layer or subassembly, two functional conducting layers surrounding or sandwiching the particular layer are needed, where the conducting layer can be a ground layer 800A, 800B and a conductive layer 804A, 804B or two conductive layers 804A, 804B.

Another potential fault is an open ground wire/ground layer connector 801A, 801B. In this situation, which can be detected by measuring the capacitance of both capacitive subassemblies which include capacitive layers 802A, 802B, the capacitance of both capacitive subassemblies cannot be accurately determined, and instead will appear to have a capacitance less than a predetermined minimum capacitance when no force is exerted on the sensor by the patient, as described above. This occurs because both capacitive layers 802A, 802B are adjacent to a ground layer 800A, 800B and ground wire, and the ground layers 800A, 800B can be electrically connected by the rivet 1108 as shown in FIGS. 11A and 11D.

Continued operation of the sensor with an open ground wire/ground layer connector 801A, 801B is possible by measuring the resistance of the resistive layer 810 between the two capacitive subassemblies using the two functional sensor wires/conductive layers connectors 806A, 806B, which surround the resistive layer 810, as shown in FIG. 8E. Measuring the resistance, which varies according to the force applied to the sensor, allows the device to determine the force applied to the sensor.

Another fault occurs when the sensor wires/conductive layers connectors 806A, 806B are shorted together. This condition is detected by measuring the resistance of the resistive layer 810 between the two capacitive subassemblies and measuring a resistance of near zero or zero. Because the sensor wires/conductive layer connectors 806A, 806B are shorted together, they cannot be used to measure the properties of the layer in between. However, the shorted sensor wires can still be used essentially as a single sensor wire/conductive layer connector 806 with the functional ground wire/ground layers connectors 801A, 801B to measure the capacitance of the capacitive subassemblies which include capacitive layers 802A, 802B, which are disposed between the ground layers 800A, 800B/ground layer connectors 801A, 801B and the sensor wires/conductive layers 804A, 804B/conductive layer connectors 806A, 806B. Because the capacitance varies with the applied force, the applied force on the sensor can be determined by measuring the capacitance of the capacitive subassemblies, which allows the continued operation of the sensor despite the sensor wires/conductive layer connectors 806A, 806B being shorted together.

Another fault is a sensor wire/conductive layer connector 806A, 806B to ground wire/ground layer connector 801A, 801B short. This fault can be detected by measuring the apparent resistance between the sensor wire/conductive layer connector 806A, 806B and the ground wire/ground layer connector 801A, 801B and obtaining a measurement of near zero or zero. For example, attempting to measure the resistance of the capacitive subassemblies which include capacitive layers 802A, 802B, which are disposed between the ground layers 800A, 800B and conductive layers 804A, 804B, will result in a resistance measurement of near zero or zero because of the short between the sensor wire/conductive layer connector 806A, 806B and ground wire/ground layer connector 801A, 801B. However, because the two sensor wires/conductive layer connectors 806A, 806B are functional, the resistance of the resistive layer 810, which is disposed between the two conductive layers 804A, 804B of the two capacitive subassemblies, can be measured. From the resistance, the force applied can be determined, which allows continued operation of the sensor despite the sensor wire to ground wire short.

FIG. 12B illustrates another embodiment of the fault detection capabilities built into the sensor and how the sensor can continue to operate despite the presence of one or more faults in a sensor having a sensor layer configuration shown in FIG. 8F and described above. One possible fault is an open sensor wire/conductive layer 804A, 804B. The open sensor wire fault can be detected by measuring the capacitance between the two resistive subassemblies and determining whether the capacitance is less than a minimum with no-force.

Continued operation of the sensor is possible by disregarding the resistance measurements from the open sensor wire and measuring the resistance of the resistive layer between the functional conductive layer/sensor wire and ground layer/ground wire. For example, with reference to FIG. 8F, if the sensor wire to conductive layer 804A is open, the resistance of the resistive layer 810A cannot be accurately determined. However, the sensor wire to conductive layer 804B is still functional, so the resistance of the resistive layer 810B can still be determined, which will allow the device to determine the force exerted on the sensor.

Another fault is an open ground wire/ground layer 800A, 800B. This fault can be detected by measuring the resistance of both resistive subassemblies and determining that the resistances of both resistive subassemblies are greater than a predetermined maximum with no-force.

Continued operation of the sensor with an open ground wire/ground layer 800A, 800B is possible by measuring the capacitance between the two resistive subassemblies. Measuring the capacitance, which varies according to the force applied to the sensor, allows the device to determine the force applied to the sensor.

Another fault occurs when the sensor wires/conductive layers 804A, 804B are shorted together. This condition is detected by measuring the resistance between the two resistive subassemblies and measuring a resistance of near zero or zero. Because the sensor wires are shorted together, they cannot be used to measure the properties of the layer in between. However, the shorted sensor wires can still be used essentially as a single sensor wire/conductive layer with the functional ground wire/ground layers 800A, 800B to measure the resistance of the resistive layers 810A, 810B, which are disposed between the ground layers 800A, 800B and the sensor wires/conductive layers 802A, 802B. Because the resistance varies with the applied force, the applied force on the sensor can be determined by measuring the resistance of the resistive layers 810A, 810B, which allows the continued operation of the sensor despite the sensor wires being shorted together.

Another fault is a sensor wire/conductive layer 802A, 802B to ground wire/ground layer 800A, 800B short. This fault can be detected by measuring the resistance between the sensor wire/conductive layer and the ground wire/ground layer and obtaining a measurement of near zero or zero. For example, attempting to measure the resistance of the resistive subassemblies, will result in a resistance measurement of near zero or zero. However, because the two sensor wires/conductive layers 804A, 804B are functional, the capacitance between the two resistive subassemblies can be measured. From the capacitance, the force applied can be determined, which allows continued operation of the sensor despite the sensor wire to ground wire short.

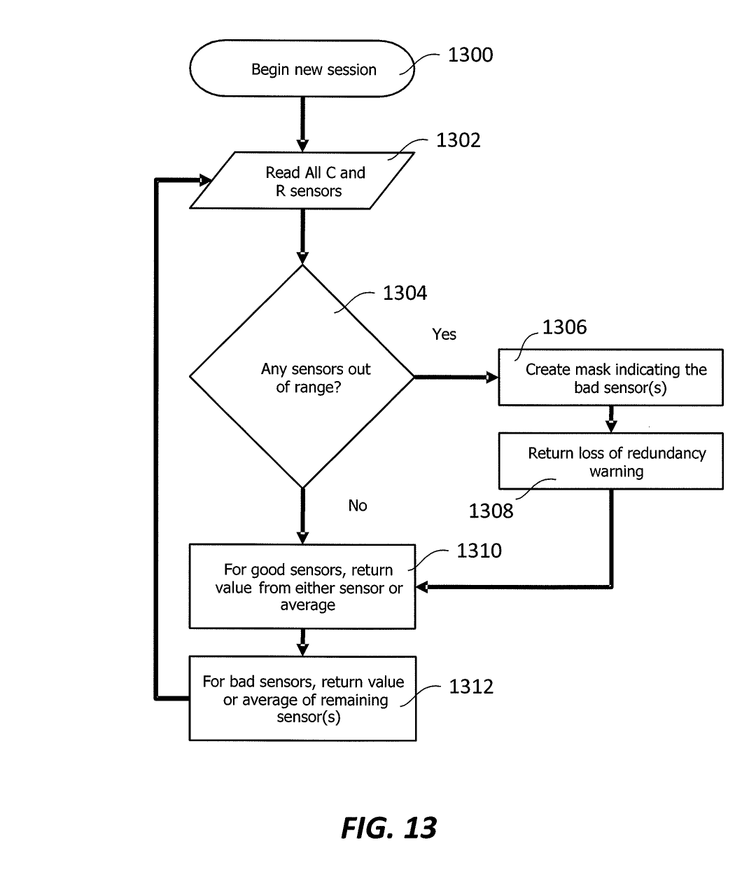

The embodiments described above in FIGS. 12A and 12B are fault tolerant because the force exerted on the sensor can be determined by a variety of means, including from either a capacitive or resistive measurement alone, as described above. In addition, FIG. 13 is a flow chart illustrating fault tolerant operation of the sensor. At step 1300 a new measurement routine is started or initiated. For example, step 1300 can represent the initialization or start up procedure when the sensor based device is put on by the patient and activated. Following initialization, the device can take measurements of the resistance of each resistive layer and the capacitance of each capacitive layer, as shown in step 1302. Once all measurements have been completed, the device compares the measurement values with the criteria set forth in FIG. 12A or 12B to determine whether there is a fault with any sensor component, as shown in step 1304. If there is a bad sensor component, such as a bad sensor wire/conductive layer or a bad ground wire/ground layer, a mask, flag or other identifier indicating that the sensor component is faulty can be assigned by the processor and stored in memory so that the device is aware that the sensor component is faulty in subsequent measurement routines, as shown in step 1306. In addition, once a faulty sensor component has been identified by the processor, a warning or alert notifying the user of the faulty sensor component and a loss of sensor redundancy can be sent to the user via a display on the device or an external display on another device, such as a personal computer, a handheld device, a tablet computer, a cell phone, a smart phone or any other device in communication with the sensor based device, as shown in step 1308.

Next, as shown in step 1310, the measurement values from the working sensor components are utilized for further analysis and processing, such as the force calculations discussed above, or the average value of the working sensor components can be used for the force calculations. Note that if no faulty sensors are detected in step 1304, the routine proceeds directly to step 1310. Following step 1310, the measurement values from the faulty sensors can be discarded or can be replaced by the values of the working sensors or an average value of the working sensors and sent for further analysis and processing, as shown in step 1312. After step 1312 is completed, a new measurement cycle can be initiated, returning the routine back to step 1302. When a plurality of sensor components are functioning properly, such as the resistance layer and the capacitive layers, improved accuracy of the force measurements can be realized by using the values from the sensor component that is expected to be most accurate. For example, the resistive layer or sensor is likely to be more accurate for light pressure or force where it will not saturate to very low resistances, while the capacitive layer or sensor is likely to be more accurate at high pressures or forces where the plates are closer together and are more sensitive to changes in the compression of the dielectric. Therefore, when light pressures or forces are measured by the sensor, the measurements from the resistive layer or sensor can be used to determine the force or pressure measurements, while when high pressures or forces are measured by the sensor, the measurements from the capacitive layer or sensor can be used to determine the force or pressure measurements.

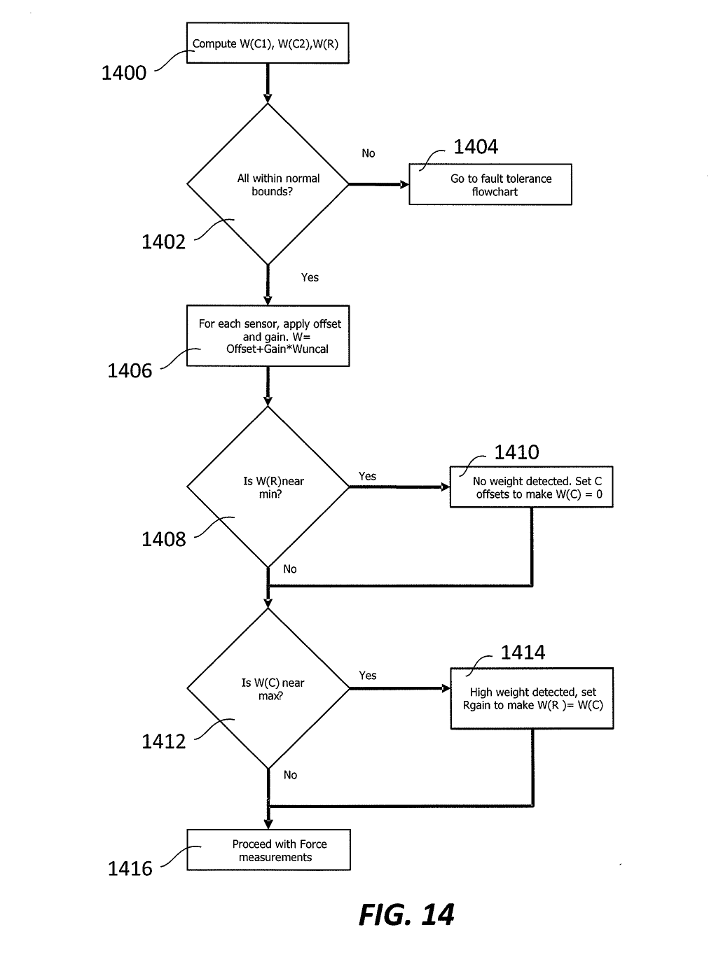

FIG. 14 is a flow chart illustrating an embodiment of a sensor auto-calibration procedure. The control unit processor of the device can compute the weight applied to the sensor components, such as the capacitive layers 802A, 802B and the resistive layer 810, when no weight or load is applied to the sensor in an unweighted or unloaded state and then when weight or load is applied to the sensor in a weighted or loaded stated, as shown in step 1400. The processor then determines whether all the weight readings are within normal bounds, as shown in step 1402. If some weight readings are not within normal bounds, the routine or procedure proceeds to the fault tolerance flow chart illustrated in FIG. 13 and described above, as shown in step 1404. If all the weight readings are within normal bounds, then for each sensor component uncalibrated weight measurement, an offset and gain is applied to the uncalibrated weight measurement in order to determine a first pass calibrated weight measurement, where the calibrated weight measurement equals the gain times the uncalibrated weight measurement plus the offset, as shown in step 1406.

The resistive layer 810, which forms a resistance sensor, very accurately measures the zero force or unweighted or unloaded threshold. This measurement can be identified as a weight at or near the minimum weight measured by the resistance sensor in the unweighted state, as shown in step 1408. The first pass calibrated weight measurement determined from the resistance sensor in the unweighted state can be used to zero the sensor and auto-calibrate the readings from the capacitive layers 802A, 802B, which form capacitance sensors. The first pass calibrated weight measurement by the capacitance sensors in the unweighted state can be set to zero by adjusting the capacitance sensor offset, as shown in step 1410. The capacitance sensor offset value that zeros the weight measurement can be stored in memory.

The capacitance sensors very accurately measure high forces because of the fixed dielectric properties that form the capacitance sensors. The first pass calibrated weight measurement by the capacitance sensors during the weighted state can be used to set the resistance sensor gain by adjusting the resistance sensor gain until the weight measurement by the resistance sensor in the weighted state equals the weight measurement by the capacitance sensors in the weighted state, as shown in steps 1412 and 1414. The adjusted resistance sensor gain can be stored in memory. In some embodiments, weight measurements by the resistance sensor are set to equal the average value of the weight measurements of the capacitance sensors. In some embodiments, the capacitance sensor gain can be determined based on the dielectric used.

After steps 1412 and 1414, the processor proceeds to calculate the force and weight measurements using the updated and auto-calibrated sensor offset and gain values. As shown in FIG. 14, the gain and offset can be set without outside calibration based on the measurements of the resistance sensor and capacitance sensor. The auto-calibration or self-calibration feature allows the sensor to compensate for changes in sensor performance or characteristics over time due to compression-set effects of the capacitive dielectric, or changes in the resistance of the resistive material due to wear and/or moisture.

In some embodiments, to determine the translation from resistance and capacitance to weight, a prototype can be built and then, known weights are applied with the resulting data entered into a table. The table can be compiled into the code running in the sensor for use in a table lookup algorithm, or a curve fit to the data and the parameters of the (typically polynomial) equation are programmed into the code. This process can be done at the factory as a factory calibration procedure. The tables and/or equations are different for resistance and capacitance. Higher weight lowers resistance and increases capacitance. The code need not directly compute resistance or capacitance. Instead, the measured parameter (from ADC or a counter) can be directly mapped to weight via a table or equation. In addition, a user calibration can be performed on the device. For example, the user can place the sensor in an unweighted state for one calibration point, and then place a known full weight onto the sensor. The user calibration can be repeated at predetermined intervals based, for example, on length of use of the device.

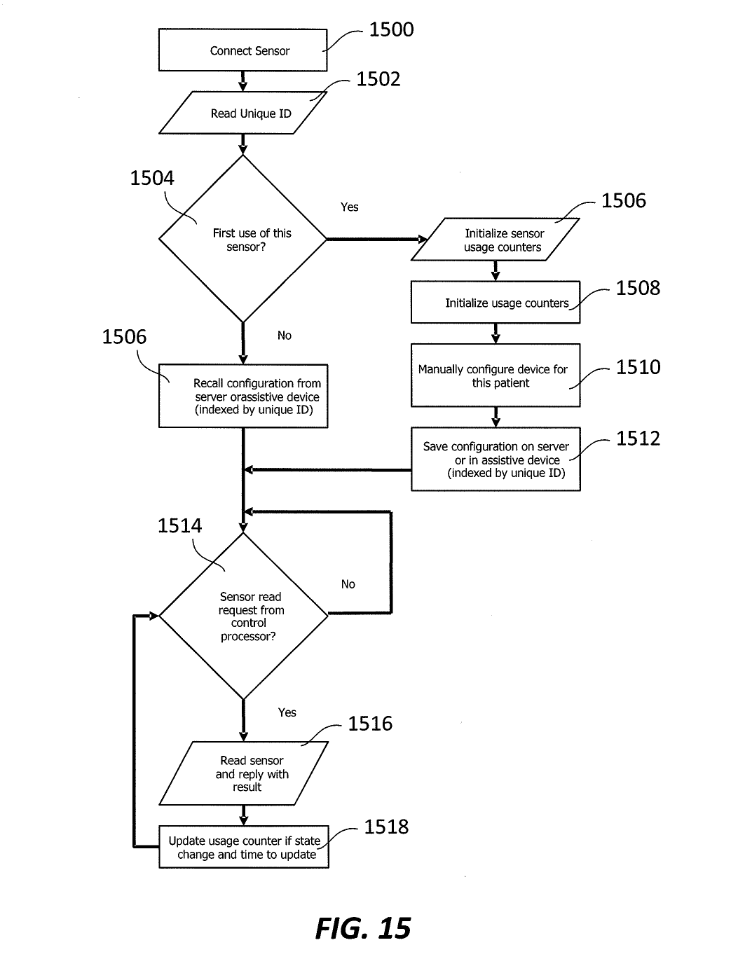

FIG. 15 is a flow chart illustrating an embodiment of sensor initialization and determining sensor end of life. The routine or procedure begins with connecting the sensor to, for example, a controller of an active orthotic device, as shown in step 1500. Next, in step 1502, the controller or processor of the controller reads a unique serial number or patient identification or other unique identifier, which will collectively be referred to as a unique ID, from the memory on the sensor PCB, as described above with reference to FIG. 10B. The controller can request the unique ID from the microcontroller on the sensor, which can transmit the unique ID upon request. Alternatively, upon connection, the microcontroller on the sensor can automatically transmit the unique ID to the controller without needing a request.

Next, in step 1504, the controller determines whether the sensor has been used before or whether this is the first use of the sensor. For example, the memory on the sensor PCB can store sensor use data that can be retrieved by or transmitted to the controller. Once this sensor use data is retrieved by the controller, the sensor use data can be stored in memory on the controller. If the controller determines that sensor use data that this is the first use of the sensor, then the controller initializes a sensor usage counter and then initializes a usage counter for the active orthotic device, as shown in steps 1506 and 1508. Next, in step 1510, the controller can prompt the user, which can be the patient or health care provider, to manually configure and customize the device for the patient. For example, the patient's weight can be input into the device along with other patient characteristics such as height, age, size, medical conditions, and rehabilitation treatment history. This information and configuration of the sensor and orthotic device case be saved as a patient profile on an external server and/or on the memory of the orthotic device itself and/or on the memory of the microcontroller of the sensor, as shown in step 1512. The patient profile can be indexed by the unique ID which enables subsequent retrieval of the patient profile to be accomplished with the unique ID. By keeping at least one copy of the patient profile settings on the external server or on the active orthotic device, the settings are not lost if the sensor is lost or if the patient leaves the sensor at home when arriving at the treatment facility for a rehabilitation session.

If in step 1504 the controller determines that the sensor has been previously used, it can recall the patient profile from the external server or from the memory of the orthotic device or sensor, as shown in step 1506. After step 1506 or 1512, the controller determines whether a sensor measurement reading is needed or should be taken, as shown in step 1514. If a sensor measurement is requested by the controller, the controller obtains a sensor measurement from the sensor as described above and then the usage counters are incremented if, for example, a state change indicates that it is time to update the count.

For example, the flash memory within the processor on the sensor PCB can be nonvolatile and can emulate an EEPROM (Electrically Erasable Programmable Read Only Memory) as described in Texas Instruments Application Note SPRAB69, which is hereby incorporated by reference in its entirety, or may contain other nonvolatile memory such as the EEPROM in the PIC family and the FRAM in some devices of the Texas Instruments family of microcontrollers and processors. The nonvolatile memory can record an activation count or step count of the sensor every N times a threshold is exceeded, or whenever commanded by the processor in the active orthotic device. For example, the counters can be incremented based on a predetermined amount of time elapsing while the sensor or orthotic device is being used, or the counter can be incremented every N times a sensor measurement cycle is completed, where N is a predetermined number that can be customized by the user or set at the factory. The sensor activation count is used to warn that the sensor end of life is approaching to help facilitate timely ordering of new sensors. The activation count can also be used to compensate for sensor wear that would otherwise make the measurements less accurate over time. For example, if the spacing of the capacitive layers decreases at a known rate or amount over time due to repeated compression, a model of the expected creep per activation or compression can be pre-programmed into the controller and the sensor measurement values can be compensated by the amount of expected creep to maintain or improve accuracy.

The above described orthosis, sensors and components provide a light weight active muscle assistance system. Although the systems have been described in considerable detail with reference to certain embodiments thereof, other versions are possible. For example, any feature disclosed in connection with any particular embodiment can be combined with any other feature disclosed in any other embodiment. Therefore, the spirit and scope of the appended claims should not be limited to the description of the exemplary versions contained herein.

What is claimed is:

1. A force sensor assembly, comprising:
a sensor for detecting force applied to a part of the body;
an interface from the sensor operable in multiple modes;
a first mode in which the interface communicates sensing information to the control system of an orthotic or prosthetic device; and
a second mode in which the interface communicates sensing information to the memory of a logging device.

2. The force sensor assembly of claim 1 in which the control system activates to apply assistance to movement in response to activation of the force sensor.

3. The force sensor assembly of claim 2 in which the sensor is a foot sensor and movement assistance is applied at the knee.

4. The force sensor assembly of claim 1 in which the logging device acquires sensing information while the sensor is disconnected from the orthotic device.

5. The force sensor assembly of claim 1 including a third mode in which the interface communicates logged information to a PC or handheld device.

6. The force sensor assembly of claim 1 in which the sensor assembly includes patient-specific configuration information.

7. The force sensor assembly of claim 1 in which the sensor assembly includes a unique identifier of the sensor.

8. The force sensor assembly of claim 1 the sensor further comprising a capacitive layer assembly having a capacitance that varies with the force applied to the part of the body and a resistive layer disposed within the capacitive layer assembly having a resistance that varies with the force applied to the part of the body and a processing unit in communication with the sensor interface configured to measure the capacitance of the capacitive layer assembly and the resistance of the resistive layer.

9. A method of sensing a force applied to a part of the body, comprising:
a first step of communicating sensing information to the control system of an orthotic or prosthetic device; and
a second step of communicating sensing information to the memory of a logging device.

10. The method of sensing the force applied as in claim 9 further comprising a third step of applying assistance to movement in response to activation of the force sensor.

11. The method of sensing the force applied as in claim 10 in which the sensor is a foot sensor and the assistance in the third step includes assistance in extending the knee joint.

12. The method of sensing the force applied as in claim 9 in which the second step is performed while the sensor is disconnected from the orthotic device.

13. The method of sensing the force applied as in claim 10 including a fourth step in which logged information is communicated to a PC or handheld device.

14. The method of sensing the force applied as in claim 13 including a fifth step of producing a patient report based on the logged information.

15. The method of sensing the force applied as in claim 14 in which the logging devices records a unique identifier of the sensor and patient-specific configuration information.
